(19) United States
(12) Patent Application Publication
(10) Pub. No.: US 2012/0259101 A1
(43) Pub. Date: Oct. 11, 2012

(54) Isopeptide Bond Formation in Bacillus Species and Uses Thereof

(76) Inventors: Li Tan, Hoover, AL (US); Charles L. Turnbough, JR., Vestavia Hills, AL (US)

(21) Appl. No.: 13/324,864

(22) Filed: Dec. 13, 2011

Related U.S. Application Data
(60) Provisional application No. 61/474,194, filed on Apr. 11, 2011.
(60) Provisional application No. 61/489,157, filed on May 23, 2011.

(51) Int. Cl. C07K 19/00

(52) U.S. Cl. 530/405

(57) ABSTRACT

A mechanism for a unique isopeptide bond formation between polypeptides is disclosed as well as sequence motifs used in such bond formation and methods of using such sequence motifs.

### BACKGROUND

1. Field of the Disclosure

The present disclosure relates generally to new mechanisms for forming specific covalent bonds between polypeptides. Specifically, the present disclosure relates to new mechanisms and sequence motifs involved in forming specific isopeptide bonds between amino acid sequences and polypeptides and uses of such sequences and polypeptides.

2. Introduction

Bacillus anthracis is a Gram-positive, aerobic soil bacterium that forms durable spores upon nutrient deprivation, and contact with these spores causes the potentially lethal disease anthrax in animals and humans (1). Formation of B. anthracis spores begins with an asymmetric septation that divides the vegetative cell into a mother cell compartment and a smaller forespore compartment, which is followed by engulfment of the forespore by the mother cell. Three protective layers called the cortex, coat, and exosporium then surround the forespore prior to mother cell lysis (2). The outermost exosporium layer, which appears to be separated from the underlying coat, is a bipartite structure consisting of a paracrystalline basal layer and an external hair-like nap (3). The filaments of the nap are formed by trimers of the collagen-like glycoprotein BclA (4-6). Recent studies suggest that BclA plays a key role in pathogenesis by promoting spore uptake by host professional phagocytic cells that carry the spores to internal tissues where spore germination and bacterial cell growth can occur (7, 8). The basal layer of the exosporium contains approximately 20 different proteins, including the proteins called BxpB, ExsY, ExsB, CotY and CotE (9). BxpB (also called ExsFA) is required for the attachment of approximately 98% of the total BclA present in the exosporium (10, 11). Attachment of the remaining BclA requires the BxpB paralog ExsFB (11).

BclA is composed of three domains: a 38-residue amino-terminal domain (NTD), an extensively glycosylated collagen-like region containing a strain-specific number of GX₁X₂ (mostly GPT) triplet amino-acid repeats, and a 134-residue carboxy-terminal domain (CTD) (5, 6, 9). The CTD is believed to function as the major nucleation site for trimerization of BclA and CTD trimers form the globular distal ends of the filaments in the nap. The highly extended collagen-like region is extensively glycosylated and its length determines the depth of the nap.

Basal layer attachment of BclA occurs through its NTD (4, 12) and deletion of the NTD prevents attachment. The attachment of BclA requires proteolytic cleavage of the NTD between residues S19 and A20 (13); however, other cleavage sites may also be recognized when the foregoing residues are absent or mutated (13). BclA attachment also involves a region of the NTD between residues 20 and 33 that includes at least one signal for the localization of BclA to the forespore (13). Proteolytic cleavage preceding NTD residue A20 occurs only after BclA is bound to the developing forespore (12). In mature spores, BclA is included in high molecular mass (>250-kDa) complexes that also include BxpB and in some cases other exosporium proteins, such as ExsY and its homolog CotY as well as ExsB and other exosporium proteins (10, 13, 14). These complexes are stable under conditions designed to dissociate non-covalently bound protein complexes and to reduce disulfide bonds (13). Furthermore, BclA is unable to form disulfide bonds with other proteins because it does not contain cysteine residues. While the art was aware that BclA is attached to the exosporium basal layer, the mechanism for attachment was not known, although it was recently suggested that the attachment occurred through a covalent bond (13).

The present disclosure demonstrates that the attachment of BclA, ExsB, CotY and ExsY and perhaps other exosporium polypeptides to the exosporium basal layer involves the formation of isopeptide bonds between an amino group of a residue on the BclA, ExsB, CotY and ExsY polypeptide and a side chain carboxyl group of an acidic residue on an acceptor protein. The identified mechanism of attachment represents a new general mechanism for attachment and cross-linking of proteins and polypeptides. The formation of the isopeptide bonds occurs through a mechanism unlike any known mechanism of protein cross-linking through isopeptide bond foimation. Donor and acceptor sequence motifs responsible for isopeptide bond formation are identified. Such donor and acceptor sequence motifs may be incorporated into polypeptides of interest in order to facilitate the specific formation of multi-polypeptide complexes and for other uses as described herein.

### BRIEF DESCRIPTION OF THE DRAWINGS

FIG. 1. shows positive ion MS/MS spectrum used to determine the sequence of a branched peptide containing BxpB residues 60-69 with AF peptides derived from the NTD of BclA attached to residues D60 and D66. The spectrum was produced by electrospray ionization collision-activated dissociation of (M+2H)²⁺ ions (m/z=728.2). Fragmentation endpoints of y-ions and b-ions are indicated on the peptide sequence. Ion labels and their meanings are: *, loss of ammonia; °, loss of water; F, loss of phenylalanine due to cleavage of the AF peptide bond; AF, loss of AF peptide due to cleavage of the isopeptide bond; multiple * and/or °, multiple losses of ammonia and/or water.

FIG. 2. shows exosporium protein complexes containing BclA NTD-eGFP fusion protein(s) attached to BxpB. After separation by sodium dodecyl sulfate (SDS)—polyacrylamide gel electrophoresis (PAGE), protein complexes were visualized by staining with Coomassie Blue and analyzed by immunoblotting with anti-GFP and anti-BxpB monoclonal antibody (MAb). Bands 1, 2, and 3 include complexes with BxpB attached to one, two, and three molecules of the BclA NTD-eGFP fusion protein, respectively. Gel locations and molecular masses of prestained protein standards are shown. The bands in the anti-GFP lane with apparent masses of approximately 30 kDa or less presumably contain free fusion protein or products of fusion protein degradation. The bands in the anti-BxpB lane with apparent masses less than that of band 1 presumably contain BxpB complexes with other basal layer proteins or free BxpB, which has a mass of 17.3 kDa.

FIG. 3. shows acidic residues of BxpB that can serve as sites for covalent attachment of BclA. Formation of >250-kDa BclA/BxpB-containing exosporium protein complexes formed by the indicated strains was detected by immunoblotting with an anti-BclA MAb. The strains examined were Sterne (WT), a Sterne mutant lacking bxpB (ΔbxpB), and variants of the ΔbxpB mutant that carried a plasmid directing the correctly timed expression of wild-type BxpB (pWT) and the indicated mutant BxpB proteins. In the 10M mutant protein, all acidic residues except D5, D12, and E14 were changed to alanines; in the 10M+D/E mutant proteins, all acidic residues except D5, D12, E14, and the indicated D/E residue were changed to alanines. Only the part of the immunoblot containing bands is shown, and the gel locations and molecular masses of prestained protein standards are indicated. The arrowhead points to the band containing glycosylated monomeric BclA, and the bracket marks the >250-kDa BclA/BxpB-containing complexes (13).

FIG. 4. shows formation of high-molecular mass complexes containing cross-linked rBclA and rBxpB. Complexes were formed in reaction mixtures containing 20 μM rBclA and 5 μM rBxpB. Samples of purified rBclA and rBxpB and of rBclA-rBxpB cross-linked complexes were separately analyzed in triplicate by SDS-PAGE. The three essentially identical gels were used to detect proteins and protein complexes by immunoblotting with either an anti-BclA or anti-BxpB MAb or by staining with Coomassie Blue

FIG. 5. shows a proposed model for the formation of isopeptide bonds that attach BclA to BxpB during exosporium assembly. (A) BclA NTD localization signals direct binding of a BclA trimer to BxpB present in the basal layer of the exosporium. (B) Each NTD of a bound BclA trimer is proteolytic cleaved between residues S19 and A20 producing a new and reactive amino terminus. The protein(s) required for cleavage remain to be identified. (C). The amino group of BclA residue A20 forms an isopeptide bond with an appropriately positioned side-chain carboxyl group of an internal BxpB acidic residue. (D) Each strand of the BclA trimer can form an isopeptide bond with one of 10 acidic residues of BxpB, with each trimer presumably attaching to three neighboring acid residues. There is no requirement, however, that all strands of the BclA trimer participate in isopeptide bond formation. The 13 acidic residues of BxpB are represented by red tick marks, and their positions within the protein are approximate.

FIG. 6 shows the amino acid sequence of BclA, BxpB, ExsY, CotY, ExsB and CotE.

FIGS. 7 A and B show exosporium protein complexes containing BclA, BxpB, ExsY, and CotY produced by wild-type and mutant B. anthracis strains. In FIG. 7A, solubilized proteins and protein complexes were separated by SDS-PAGE and visualized by immunoblotting with anti-BxpB and anti-ExsY/CotY MAbs (the latter MAb reacts equally with ExsY and CotY). In the anti-BxpB blot, equivalent samples of wild-type (WT), ΔcotY, ΔexsY, ΔexsYΔcotY (dblΔ) spores along with purified rBxpB were analyzed (Lane 1, WT; Lane 2, ΔcotY; Lane 3, ΔexsY; Lane 4, ΔexsYΔcotY (dblΔ); Lane 5, purified purified rBxpB). The arrowhead points to a band presumed to contain a BxpB/ExsY heterodimer. Gel locations and molecular masses of prestained protein standards are indicated. The brace indicates the position of >250-kDa exosporium protein complexes. In the anti-CotY/ExsY blot, the same spore samples along with an equivalent sample of ΔbxpB spores were analyzed (Lane 1, WT; Lane 2, ΔcotY; Lane 3, ΔexsY; Lane 4, ΔexsYΔcotY (dblΔ); Lane 5, ΔbxpB) In FIG. 7B, spore-free material in washes used to collect wild-type and the indicated mutant spores from solid medium were analyzed as above, except that proteins were visualized by immunoblotting them with anti-BxpB and anti-BclA MAbs. Only the parts of the immunoblots containing bands are shown. Lane 1, WT; Lane 2, ΔcotY; Lane 3, ΔexsY; Lane 4, ΔexsYΔcotY (dblΔ). The brace marks the >250-kDa BclA/BxpB/ExsY/CotY-containing complexes. In all immunoblots, gel locations and molecular masses of prestained protein standards are indicated.

FIG. 8 shows formation of isopeptide bonds involving acidic residues of BxpB and amino-terminal residues of ExsY, CotY, and BclA. The 13 acidic residues of BxpB, which contains 167 amino acids, are represented by tick marks in the figure. ExsY, CotY, and BxpB are represented by symbols according to the legend. The symbol for each protein is positioned above the BxpB acidic residues with which that protein can participate in isopeptide bond formation. Multiple symbols above a tick mark indicate that each of the proteins symbolized react separately at this position.

FIG. 9 shows formation of isopeptide bonds involving acidic residues of ExsY and CotY and amino-terminal residues of ExsY, CotY, and ExsB. ExsY and CotY contain 15 and 18 acidic residues (out of 152 and 156 amino acids), respectively, which are represented by tick marks in the figure. ExsY, CotY, and ExsB are represented by symbols according to the legend. The symbol for each protein is positioned above the ExsY/CotY acidic residues with which that protein can participate in isopeptide bond formation. Multiple symbols above a tick mark indicate that each of the proteins symbolized react separately at this position. The absence of a protein symbol above a tick mark indicates that isopeptide bond formation at this site was not observed with the branched peptides analyzed in this study.

FIG. 10 shows formation of isopeptide bonds involving acidic residues of CotE and amino-terminal residues of ExsY, CotY, and ExsB. The 38 acidic residues of CotE, which contains 180 amino acids, are represented by tick marks in the figure. ExsY, CotY, and ExsB are represented by symbols according to the legend. The symbol for each protein is positioned above the CotE acidic residues with which that protein can participate in isopeptide bond formation. Multiple symbols above a tick mark indicate that each of the proteins symbolized react separately at this position. The absence of a protein symbol above a tick mark indicates that isopeptide bond formation at this site was not observed with the branched peptides analyzed in this study.

FIG. 11 shows a model for the exosporium protein network cross-linked by isopeptide bonds during exosporium assembly. At the outer surface of the basal layer, BclA trimers form isopeotide bonds with all regions of BxpB except its amino-terminal domain, which is cross-linked by ExsY and CotY as donor proteins. Within the basal layer, ExsY and CotY also act as acceptor proteins to cross-link with the amino-termini of ExsB and of separate molecules of ExsY and CotY. Furthermore, ExsY, CotY, and ExsB act as donor proteins to attach to acidic residues of CotE. CotE, which is a morphogenetic protein located at the inner surface of basal layer, presumably connects the exosporium to the spore coat in an undetermined manner. In summary, BclA and ExsB function only as donor proteins, BxpB and CotE function only as acceptor proteins, and ExsY and CotY perform both functions.

### DETAILED DESCRIPTION

Isopeptide bonds are protein modifications found throughout nature in which amide linkages are formed between functional groups of two amino acids with at least one of the functional groups provided by an amino acid side-chain. Isopeptide bonds generate cross-links within and between proteins that are necessary for proper protein structure and function. In the present disclosure it is shown that BclA, the dominant structural protein of the external nap of B. anthraces spores, is attached to the underlying exosporium basal layer protein BxpB via isopeptide bonds formed through a mechanism fundamentally different from previously described mechanisms of isopeptide bond formation. Features of this mechanism are the generation of a reactive amino group by proteolytic cleavage and promiscuous selection of acidic side-chains. This mechanism, which relies only on short peptide sequences in protein substrates, could be a general mechanism in vivo and adapted for protein cross-linking in vitro. In addition, CotY, ExsY, ExsB and CotE are shown to participate in isopeptide bond formation as well.

The outermost exosporium layer of B. anthracis spores, the causative agents of anthrax, is comprised of a basal layer and an external hair-like nap. The nap includes filaments composed of trimers of the collagen-like glycoprotein BclA. Essentially all BclA trimers are tightly attached to the spore in a process requiring the basal layer protein BxpB (also called ExsFA). Both BclA and BxpB are incorporated into stable high-molecular-mass complexes, suggesting that BclA is attached directly to BxpB. The 38-residue amino-terminal domain of BclA, which is normally proteolytically cleaved between residues 19 and 20, is necessary and sufficient for basal layer attachment. In the present disclosure, we demonstrate that BclA attachment occurs through the formation of isopeptide bonds between the free amino group of the NTD of BclA and a side-chain carboxyl group of an acidic residue of BxpB. In one embodiment, the residue A20; in another embodiment, the residue is F21 or V26. Ten of the 13 acidic residues of BxpB can participate in isopeptide bond formation, and at least three BclA polypeptide chains can be attached to a single molecule of BxpB. The present disclosure also demonstrates that similar cross-linking occurs in vitro between purified recombinant BclA and BxpB, indicating that the reaction is spontaneous. Furthermore, the present disclosure shows isopeptide bond formation between the polypeptide pairs shown in Table 4. The mechanism of isopeptide bond formation, specifically the formation of a reactive amino group by proteolytic cleavage and the promiscuous selection of side-chain carboxyl groups of internal acidic residues, appears to be different from other known mechanisms for protein cross-linking through isopeptide bonds. Analogous mechanisms appear to be involved in cross-linking other spore proteins and could be found in unrelated organisms.

### Donor and Acceptor Sequence Motifs

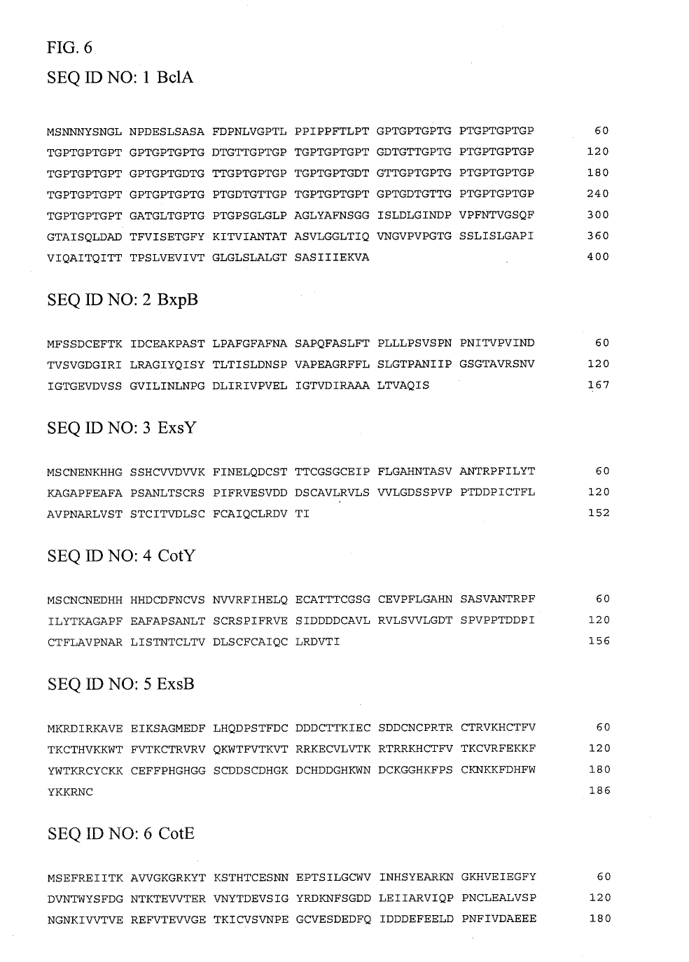

The present disclosure demonstrates that sequence motifs present in the exosporium of B. anthracis, such as, but not limited to, the BclA, BxpB, ExsB, CotE, CotY and ExsY polypeptides, are sufficient to direct formation of isopeptide bonds both in vivo and in vitro. Sequence motifs have been identified that are responsible for isopeptide bond formation. Such sequence motifs may be used as described herein. In one embodiment, such sequence motifs are incorporated into polypeptides of interest and used as described herein. The sequence motifs described include both donor sequences (those sequences that donate the alpha-amino group) and acceptor sequences (those sequences that provide the side chain group, such as a carboxyl group from an acidic amino acid such as, but not limited to, glutamate or aspartate). The BclA, CotY, ExsY and ExsB polypeptides have been demonstrated to contain donor sequences. The BxpB, CotY, ExsY and CotE polypeptides have been demonstrated to contain acceptor sequences. Note that the CotY and ExsY polypeptides contain both donor and acceptor sequences. The amino acid sequences for BclA, BxpB, ExsY, CotY, ExsB and CotE are shown in FIG. 6 and designated SEQ ID NOS: 1-6, respectively.

### Donor Sequence Motifs

In one embodiment, the donor sequence consists of, consists essentially of or comprises a sequence of at least 5, at least 10, at least 15, at least 20 or at least 25 residues from the sequence of SEQ ID NOS: 1, 3, 4 or 5. In another embodiment, the donor sequence consists of, consists essentially of or comprises a sequence of at least 5, at least 10, at least 15, at least 20 or at least 25 residues from the first 50 residues from the sequence of SEQ ID NOS: 1, 3, 4 or 5. In yet another embodiment, the donor sequence consists of, consists essentially of or comprises a sequence of at least 5, at least 10, at least 15, at least 20 or at least 25 residues from first 40 residues from the sequence of SEQ ID NOS: 1, 3, 4 or 5. In still another embodiment, the donor sequence consists of, consists essentially of or comprises a sequence of at least 5, at least 10, at least 15, at least 20 or at least 25 residues from first 30 residues from the sequence of SEQ ID NOS: 1, 3, 4 or 5. In still another embodiment, the donor sequence consists of consists essentially of or comprises a sequence at least 80% identical, 90% identical, 95% identical or 99% identical to the sequences described above. In one embodiment of the foregoing, the recited amino acid residues are contiguous amino acid residues; in an alternate embodiment, the recited amino acid residues are non-contiguous amino acid residues. In any of the foregoing, the initiating methionine residue may be removed, if present.

In one embodiment, the donor sequence consists of, consists essentially of or comprises a sequence of 5 or less, 10 or less, 15 or less, 20 or less or 25 or less residues from the sequence of SEQ ID NOS: 1, 3, 4 or 5. In another embodiment, the donor sequence consists of, consists essentially of or comprises a sequence of 5 or less, 10 or less, 15 or less, 20 or less or 25 or less residues from the first 50 residues from the sequence of SEQ ID NOS: 1, 3, 4 or 5. In yet another embodiment, the donor sequence consists of, consists essentially of or comprises a sequence of 5 or less, 10 or less, 15 or less, 20 or less or 25 or less residues from first 40 residues from the sequence of SEQ ID NOS: 1, 3, 4 or 5. In still another embodiment, the donor sequence consists of, consists essentially of or comprises a sequence of 5 or less, 10 or less, 15 or less, 20 or less or 25 or less from first 30 residues from the sequence of SEQ ID NOS: 1, 3, 4 or 5. In still another embodiment, the donor sequence consists of, consists essentially of or comprises a sequence at least 80% identical, 90% identical, 95% identical or 99% identical to the sequences described above. In one embodiment of the foregoing, the recited amino acid residues are contiguous amino acid residues; in an alternate embodiment, the recited amino acid residues are non-contiguous amino acid residues. In any of the foregoing, the initiating methionine residue may be removed, if present.

In one embodiment, the donor sequence consists of, consists essentially of or comprises the NTD of the polypeptides disclosed in SEQ ID NOS: 1, 3, 4 or 5.

In one embodiment, the donor sequence is an amino acid sequence from the BclA polypeptide. In a specific embodiment, the donor sequence may be from the NTD domain of BclA. In such an embodiment, the donor sequence may be contained in amino acid residues 1-40, 1-38, 1 and 20-38, 20-33, 20-38, 10-35 or 20-35 of SEQ ID NO: 1. In a further embodiment, the donor sequence consists of, consists essentially of or comprises a sequence of at least 5, at least 10, at least 15, at least 20 or at least 25 residues from amino acids 1-40, 1-38, 1 and 20-38, 20-33, 20-38, 10-35 or 20-35 of SEQ ID NO: 1. In a further embodiment, the donor sequence consists of, consists essentially of or comprises a sequence of 5 or less, 10 or less, 15 or less, 20 or less or 25 or less residues from amino acids 1-40, 1-38, 1 and 20-38, 20-33, 20-38, 10-35 or 20-35 of SEQ ID NO: 1. In the foregoing, the recited amino acid residues are contiguous amino acid residues; in an alternate embodiment, the recited amino acid residues are non-contiguous amino acid residues. In another specific embodiment, the donor sequence is the full length amino acid sequence of the BclA polypeptide. In still another specific embodiment, the donor sequence is the full length amino acid sequence of the BclA polypeptide minus the initiating methionine residue. In one embodiment of the foregoing, the donor sequence contains a reactive alpha amino group. In any of the foregoing, the initiating methionine residue may be removed, if present.

Non-limiting examples of exemplary donor sequences include from BclA include, but are not limited to an amino acid sequence consisting of, consisting essentially of or comprising the following: 1) AFDPNLVGPTLPPIPPFTL; 2) AFDPNLVGPTLPPI; 3) FDPNLVGPTLPPI; 4) AFDPNLPPI; 5) FDPNLPPI; 6) LVGPTLPPI; 7) VGPTLPPI; 8) Xaa₍₁₋₅₎LVGPTLPPIXaa₍₀₋₅₎; 9) Xaa₍₁₋₆₎VGPTLPPIXaa₍₀₋₅₎; (SEQ ID NOS: 7-15) (where X can be any amino acid). In addition, fragments of 5 or more or 10 or more of the above-disclosed amino acid sequences may be used.

In still another embodiment, the donor sequence from BclA consists of, consists essentially of or comprises a sequence at least 80% identical, 90% identical, 95% identical or 99% identical to the sequences described above.

In another embodiment, the donor sequence is from the ExsB polypeptide. In such an embodiment, the donor sequence may be contained in amino acid residues 1-40, 20-38, 20-30, 10-35 or 20-35 of SEQ ID NO: 5. In a further embodiment, the donor sequence consists of, consists essentially of or comprises a sequence of at least 5, at least 10 or at least 15 residues from amino acids 1-40, 20-38, 20-30, 10-35 or 20-35 of SEQ ID NO: 5. In a further embodiment, the donor sequence consists of, consists essentially of or comprises a sequence of 5 or less, 10 or less, 15 or less, 20 or less or 25 or less residues from amino acids 1-40, 20-38, 20-30, 10-35 or 20-35 of SEQ ID NO: 5. In the foregoing, the recited amino acid residues are contiguous amino acid residues; in an alternate embodiment, the recited amino acid residues are non-contiguous amino acid residues. In another specific embodiment, the donor sequence is the full length amino acid sequence of the ExsB polypeptide. In still another specific embodiment, the donor sequence is the full length amino acid sequence of the ExsB polypeptide minus the initiating methionine residue. In one embodiment of the foregoing, the donor sequence comprises a reactive alpha amino group. In any of the foregoing, the initiating methionine residue may be removed, if present.

Non-limiting examples of exemplary donor sequences include from ExsB include, but are not limited to an amino acid sequence consisting of, consisting essentially of or comprising the following: 1) XₐKRDIRKAVEEIKSAGMEDFLHQDPSTFDC; 2) VE EIKSAGMEDFLHQDPSTF; 3) KSAGMEDFLHQ; (SEQ ID NOS: 16-18) (where X can be any amino acid). In addition, fragments of 5 or more or 10 or more of the above-disclosed amino acid sequences may be used.

In still another embodiment, the donor sequence from ExsB consists of, consists essentially of or comprises a sequence at least 80% identical, 90% identical, 95% identical or 99% identical to the sequences described above.

In another embodiment, the donor sequence is from the ExsY polypeptide. In such an embodiment, the donor sequence may be contained in amino acid residues 1-40, 1-30, 1-20, 1-10, or 1-5 of SEQ ID NO: 3. In a further embodiment, the donor sequence consists of, consists essentially of or comprises a sequence of at least 5, at least 10 or at least 15 residues from amino acids 1-40, 1-30, 1-20, 1-10, or 1-5 of SEQ ID NO: 3. In a further embodiment, the donor sequence consists of, consists essentially of or comprises a sequence of 5 or less, 10 or less, 15 or less, 20 or less or 25 or less residues from amino acids 1-40, 1-30, 1-20, 1-10, or 1-5 of SEQ ID NO: 3. In the foregoing, the recited amino acid residues are contiguous amino acid residues; in an alternate embodiment, the recited amino acid residues are non-contiguous amino acid residues. In another specific embodiment, the donor sequence is the full length amino acid sequence of the ExsY polypeptide. In still another specific embodiment, the donor sequence is the full length amino acid sequence of the ExsY polypeptide minus the initiating methionine residue. In one embodiment of the foregoing, the donor sequence comprises a reactive alpha amino group. In any of the foregoing, the initiating methionine residue may be removed, if present.

Non-limiting examples of exemplary donor sequences include from ExsY include, but are not limited to an amino acid sequence consisting of, consisting essentially of or comprising the following: 1) XₐSCNENKHHGSSHCVVDVVK; 2) XₐSCNENK; 3) XₐSCNENKHHGSS; or 4) XₐSCNENKHHGSSHCVVD (SEQ ID NOS: 20-24) (where X can be absent or any amino acid). In addition, fragments of 5 or more or 10 or more of the above-disclosed amino acid sequences may be used.

In still another embodiment, the donor sequence from ExsY consists of, consists essentially of or comprises a sequence at least 80% identical, 90% identical, 95% identical or 99% identical to the sequences described above.

In another embodiment, the donor sequence is from the CotY polypeptide, including a full length CotY polypeptide. In such an embodiment, the donor sequence may be contained in amino acid residues 1-40, 1-30, 1-20, 1-10, or 1-5 of SEQ ID NO: 4. In a further embodiment, the donor sequence consists of, consists essentially of or comprises a sequence of at least 5, of at least 10 or at least 15 residues from amino acids 1-40, 1-30, 1-20, 1-10, or 1-5 of SEQ ID NO: 4. In a further embodiment, the donor sequence consists of, consists essentially of or comprises a sequence of 5 or less, 10 or less, 15 or less, 20 or less or 25 or less residues from amino acids 1-40, 1-30, 1-20, 1-10, or 1-5 of SEQ ID NO: 4. In the foregoing, the recited amino acid residues are contiguous amino acid residues; in an alternate embodiment, the recited amino acid residues are non-contiguous amino acid residues. In another specific embodiment, the donor sequence is the full length amino acid sequence of the CotY polypeptide. In still another specific embodiment, the donor sequence is the full length amino acid sequence of the CotY polypeptide minus the initiating methionine residue. In one embodiment of the foregoing, the donor sequence comprises a reactive alpha amino group. In any of the foregoing, the initiating methionine residue may be removed, if present.

Non-limiting examples of exemplary donor sequences include from CotY include, but are not limited to an amino acid sequence consisting of, consisting essentially of or comprising the following: 1) XₐSCNCNEDHHHHDCDFNCVS; 2) XₐSCNCNE; 3) XₐSCNCNEDHHHH; or 4) XₐSCNCNEDHHHHDCDFN (SEQ ID NOS; 23-26) (where X can be absent or any amino acid). In addition, fragments of 5 or more or 10 or more of the above-disclosed amino acid sequences may be used.

In still another embodiment, the donor sequence from CotY consists of, consists essentially of or comprises a sequence at least 80% identical, 90% identical, 95% identical or 99% identical to the sequences described above.

In any of the foregoing donor sequences, the donor sequence disclosed may be contained in a larger polypeptide sequence. The larger polypeptide sequence in one embodiment is a polypeptide sequence not associated with the donor sequences in vivo. Furthermore, in any of the foregoing donor sequences, the donor sequence disclosed may be modified by cleavage of the donor sequence. Any cleavage mechanisms known in the art may be used, including but not limited to, cleavage by a restriction endonuclease.

One or more donor sequences may be incorporated into a polypeptide of interest for use as described herein. The donor sequences described herein may be derived from naturally occurring polypeptides described herein or may be manufactured by means known in the art.

### Acceptor Sequence Motifs

In one embodiment, the acceptor sequence consists of, consists essentially of or comprises a sequence of at least 10, at least 30, at least 50 or at least 100 residues from the sequence of SEQ ID NOS: 2, 3, 4 or 6. In another embodiment, the acceptor sequence consists of, consists essentially of or comprises a sequence of at least 5, at least 15, at least 20 or at least 25 residues from the sequence of SEQ ID NOS: 2, 3, 4 or 6. In another embodiment, the donor sequence consists of, consists essentially of or comprises a sequence of at least 5, at least 10, at least 15, at least 20 or at least 25 residues around any acidic amino acid residue from the sequence of SEQ ID NOS: 1, 3, 4 or 5. In the foregoing, the recited amino acid residues are contiguous amino acid residues; in an alternate embodiment, the recited amino acid residues are non-contiguous amino acid residues.

In one embodiment, the acceptor sequence consists of, consists essentially of or comprises a sequence of 10 or less, 30 or less, 50 or less or 100 or less residues from the sequence of SEQ ID NOS: 2, 3, 4 or 6. In another embodiment, the acceptor sequence consists of, consists essentially of or comprises a sequence of 5 or less, 15 or less, 20 or less or 25 or less residues from the sequence of SEQ ID NOS: 2, 3, 4 or 6. In another embodiment, the donor sequence consists of consists essentially of or comprises a sequence of 5 or less, 15 or less, 20 or less or 25 or less residues around any acidic amino acid residue from the sequence of SEQ ID NOS: 1, 3, 4 or 5. In the foregoing, the recited amino acid residues are contiguous amino acid residues; in an alternate embodiment, the recited amino acid residues are non-contiguous amino acid residues.

In one embodiment, the acceptor sequence is from the BxpB polypeptide. In another embodiment, the acceptor sequence is the full length BxpB polypeptide or the full length BxpB polypeptide minus the initiating methionine residue. In a further embodiment, the acceptor sequence consists of, consists essentially of or comprises a sequence shown in Tables 1-3 of the present disclosure (SEQ ID NOS. 27-63). In another embodiment, the acceptor sequence consists of, consists essentially of or comprises at least 5, at least 10, at least 20 or at least 30 amino acid residues immediately left and/or right of residue D5, D12, D60, D66, D87, D127, D141, D155, E7, E14 E94, E125, E149, (with reference to SEQ ID NO: 2). In another embodiment, the acceptor sequence consists of, consists essentially of or comprises 5 or less, 10 or less, 20 or less or 30 or less amino acid residue immediately left and/or right of residue D5, D12, D60, D66, D87, D127, D141, D155, E7, E14 E94, E125, E149, (with reference to SEQ ID NO: 2). In another embodiment, the acceptor sequence consists of, consists essentially of or comprises at least 5, at least 10, at least 20 or at least 30 amino acid residues immediately left and/or right of residue D87, E94, E125 or D127 (with reference to SEQ ID NO: 2). In another embodiment, the acceptor sequence consists of, consists essentially of or comprises at least 5, at least 10, at least 20 or at least 30 amino acid residues immediately left and/or right of residue E125 or D127 (with reference to SEQ ID NO: 2). In another embodiment, the acceptor sequence consists of consists essentially of or comprises 5 or less, 10 or less, 20 or less or 30 or less amino acid residues immediately left and/or right of residue D87, E94, E125 or D127 (with reference to SEQ ID NO: 2). In another embodiment, the acceptor sequence consists of, consists essentially of or comprises 5 or less, 10 or less, 20 or less or 30 or less amino acid residues immediately left and/or right of residue E125 or D127 (with reference to SEQ ID NO: 2).

In one embodiment, the acceptor sequence is from the CotE polypeptide. In another embodiment, the acceptor sequence is the full length CotE polypeptide or the full length CotE polypeptide minus the initiating methionine residue. In another embodiment, the acceptor sequence consists of, consists essentially of or comprises at least 5, at least 10, at least 20 or at least 30 amino acid residues immediately left and/or right of residue D61, D69, D85, D93, D99, D100, D156, D158, D162, D163, D164, D170, D176, E3, E6, E27, E31, E46, E55, E57, E75, E79, E86, E102, E115, E130, E132, E136, E140, E150, E154, E157, E165, E167, E168, E178, E179 or E180 (with reference to SEQ ID NO: 6). In another embodiment, the acceptor sequence consists of, consists essentially of or comprises at least 5, at least 10, at least 20 or at least 30 amino acid residues immediately left and/or right of residue D61, D69, D85, D93, D99, D100, E3, E6, E27, E31, E46, E55, E57, E75, E79, E86, E102, E115, E130, E132, E136, E140 or E154 (with reference to SEQ ID NO: 6). In another embodiment, the acceptor sequence consists of, consists essentially of or comprises 5 or less, 10 or less, 20 or less or 30 or less amino acid residues immediately left and/or right of residue D61, D69, D85, D93, D99, D100, D156, D158, D162, D163, D164, D170, D176, E3, E6, E27, E31, E46, E55, E57, E75, E79, E86, E102, E115, E130, E132, E136, E140, E150, E154, E157, E165, E167, E168, E178, E179 or E180 (with reference to SEQ ID NO: 6). In another embodiment, the acceptor sequence consists of, consists essentially of or comprises 5 or less, 10 or less, 20 or less or 30 or less amino acid residues immediately left and/or right of residue D61, D69, D85, D93, D99, D100, E3, E6, E27, E31, E46, E55, E57, E75, E79, E86, E102, E115, E130, E132, E136, E140 or E154 (with reference to SEQ ID NO: 6). In another embodiment, the acceptor sequence consists of, consists essentially of or comprises at least 5, at least 10, at least 20 or at least 30 amino acid residues immediately left and/or right of residue E46, E55, E57, E79 or E115 (with reference to SEQ ID NO: 6). In another embodiment, the acceptor sequence consists of, consists essentially of or comprises 5 or less, 10 or less, 20 or less or 30 or less amino acid residues immediately left and/or right of residue E46, E55, E57, E79 or E115 (with reference to SEQ ID NO: 6).

In one embodiment, the acceptor sequence is from the CotY polypeptide. In another embodiment, the acceptor sequence is the full length CotY polypeptide or the full length CotY polypeptide minus the initiating methionine residue. In another embodiment, the acceptor sequence consists of, consists essentially of or comprises at least 5, at least 10, at least 20 or at least 30 amino acid residues immediately left and/or right of residue D8, D13, D15, D93, D94, D95, D96, D109, D117, D118, D141, D153, E7, E28, E31, E42, E71 or E90 (with reference to SEQ ID NO: 4). In another embodiment, the acceptor sequence consists of consists essentially of or comprises at least 5, least 10, at least 20 or at least 30 amino acid residues immediately left and/or right of residue D8, D13, D15, D95, D141, E7, E71 or E90 (with reference to SEQ ID NO: 4). In another embodiment, the acceptor sequence consists of, consists essentially of or comprises 5 or less, 10 or less, 20 or less or 30 or less amino acid residues immediately left and/or right of residue D8, D13, D15, D93, D94, D95, D96, D109, D117, D118, D141, D153, E7, E28, E31, E42, E71 or E90 (with reference to SEQ ID NO: 4). In another embodiment, the acceptor sequence consists of, consists essentially of or comprises 5 or less, 10 or less, 20 or less or 30 or less amino acid residues immediately left and/or right of residue D8, D13, D15, D95, D141, E7, E71 or E90 (with reference to SEQ ID NO: 4). In another embodiment, the acceptor sequence consists of, consists essentially of or comprises at least 5, at least 10, at least 20 or at least 30 amino acid residues immediately left and/or right of residue D141, E7 or E71 (with reference to SEQ ID NO: 4). In another embodiment, the acceptor sequence consists of, consists essentially of or comprises 5 or less, 10 or less, 20 or less or 30 or less amino acid residues immediately left and/or right of residue D141, E7 or E71 (with reference to SEQ ID NO: 4).

In one embodiment, the acceptor sequence is from the ExsY polypeptide. In another embodiment, the acceptor sequence is the full length ExsY polypeptide or the full length ExsY polypeptide minus the initiating methionine residue. In another embodiment, the acceptor sequence consists of, consists essentially of or comprises at least 5, at least 10, at least 20 or at least 30 amino acid residues immediately left and/or right of residue D17, D27, D89, D90, D91, D105, D113, D114, D137, D149, E5, E24, E38, E67 or E86 (with reference to SEQ ID NO: 3). In another embodiment, the acceptor sequence consists of, consists essentially of or comprises at least 5, at least 10, at least 20 or at least 30 amino acid residues immediately left and/or right of residue D17, D27, D89, D137, E24, E38, E67 or E86 (with reference to SEQ ID NO: 3). In another embodiment, the acceptor sequence consists of consists essentially of or comprises 5 or less, 10 or less, 20 or less or 30 or less amino acid residues immediately left and/or right of residue D17, D27, D89, D90, D91, D105, D113, D114, D137, D149, E5, E24, E38, E67 or E86 (with reference to SEQ ID NO: 3). In another embodiment, the acceptor sequence consists of, consists essentially of or comprises 5 or less, 10 or less, 20 or less or 30 or less amino acid residues immediately left and/or right of residue D17, D27, D89, D137, E24, E38, E67 or E86 (with reference to SEQ ID NO: 3). In another embodiment, the acceptor sequence consists of, consists essentially of or comprises at least 5, at least 10, at least 20 or at least 30 amino acid residues immediately left and/or right of residue D17, D27, D89, D137, E38, E67 or E86 (with reference to SEQ ID NO: 3). In another embodiment, the acceptor sequence consists of, consists essentially of or comprises at least 5, at least 10, at least 20 or at least 30 amino acid residues immediately left and/or right of residue D27 (with reference to SEQ ID NO: 3). In another embodiment, the acceptor sequence consists of, consists essentially of or comprises 5 or less, 10 or less, 20 or less or 30 or less amino acid residues immediately left and/or right of residue D17, D27, D89, D137, E38, E67 or E86 (with reference to SEQ ID NO: 3). In another embodiment, the acceptor sequence consists of, consists essentially of or comprises 5 or less, 10 or less, 20 or less or 30 or less amino acid residues immediately left and/or right of residue D27 (with reference to SEQ ID NO: 3).

In any of the foregoing acceptor sequences, the acceptor sequences disclosed may be contained in a larger polypeptide sequence. The larger polypeptide sequence in one embodiment is a polypeptide sequence not associated with the acceptor sequences in vivo.

One or more acceptor sequences may be incorporated into a polypeptide of interest for use as described herein. The acceptor sequences described herein may be derived from naturally occurring polypeptides described herein or may be manufactured by means known in the art.

Furthermore, one of skill will recognize that individual substitutions, deletions or additions which alter, add or delete a single amino acid or a small percentage of amino acids (typically less than about 5%, more typically less than about 1%) in an encoded sequence are conservatively modified variations where the alterations result in the substitution of an amino acid with a chemically similar amino acid. Conservative substitution tables providing functionally similar amino acids are well known in the art.

The following example groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” also refers to utilizing a substituting amino acid which is identical to the amino acid being substituted except that a functional group in the side chain is protected with a suitable protecting group. The donor and acceptor sequences described above also include all of the foregoing with conservative amino acid substitutions.

### Combinations of Donor and Acceptor Sequences

The donor and acceptor sequences disclosed herein have been demonstrated to have broad reactivity to one another. In in vivo experiments, certain selectivity between donor and acceptor sequences has been demonstrated. For example, see Examples 1 and 2. However, this selectivity was shown not to exist in the in vitro situation (see Example 4).

Therefore, the present disclosure provides combinations of donor and acceptor sequences capable of reacting with one another to form a covalent bond, such as an isopeptide bond. In one embodiment, the donor/acceptor sequence pair comprises any donor sequence disclosed herein in combination with any acceptor sequence disclosed herein.

As discussed herein, any of the foregoing donor and/or acceptor sequences may be contained in a larger polypeptide sequence. The larger polypeptide sequence in one embodiment is a polypeptide sequence not associated with the donor and/or acceptor sequences in vivo. Furthermore, in any of the foregoing donor sequences, the donor sequence disclosed may be modified by cleavage of the donor sequence. Any cleavage mechanisms known in the art may be used, including but not limited to, cleavage by a restriction endonuclease. For example, the donor sequence may be cleaved to remove one or more N-terminal amino acids.

One or more donor and/or acceptor sequences may be incorporated into a polypeptide of interest for use as described herein. The donor and/or acceptor sequences described herein may be derived from naturally occurring polypeptides described herein or may be manufactured by means known in the art.

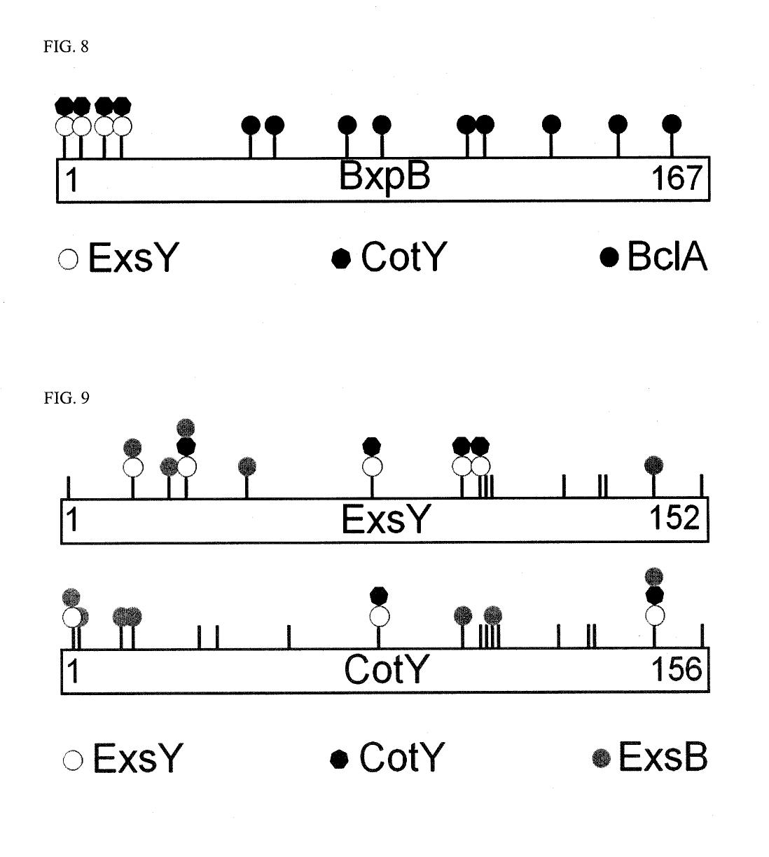

In one embodiment, the donor and acceptor sequences are sequences shown to form covalent bonds as disclosed in Tables 1-3 and 5-10, FIGS. 8-10 and in the present specification. For example, as shown in Table 3 row 1, the donor sequence is the NTD of the BclA and the acceptor sequence is residues 1-10 of BxpB. Furthermore, the donor and acceptor sequences are sequences around the specific amino acid residues shown to form covalent bonds as disclosed in Table 4 and in the present specification. For example, as shown in Table 4, the donor sequence is an amino terminal sequence of the CotY protein and the acceptor sequence is an amino acid sequence containing D5, D12, E7 or E14 of BxpB; further examples are provided in Table 5-10. As discussed above, such acceptor sequence may contain a specified number of residues on the left and/or right (such as, but not limited to, at least 5, at least 10, at least 20 or at least 30 amino acid residues immediately left and/or right or 5 or less, 10 or less, 20 or less or 30 or less amino acid residues immediately left and/or right) of the specified residue or be the full length polypeptide.

In another embodiment, the donor sequence is an amino terminal sequence of the BclA polypeptide or the full length BclA polypeptide and the acceptor sequence is an amino acid sequence containing: (i) at least one amino acid selected from the group consisting of D5, D12, D60, D66, D87, D127, D141, D155, E7, E14, E94, E125 and E149 of BxpB; (ii) at least one amino acid selected from the group consisting of D60, D66, D87, D127, D155, E94, E125 and E149 of BxpB; (iii) at least one amino acid selected from the group consisting of D66, D87, D127, D141, D155, E7, E94 and E125 of BxpB; and/or (iv) at least one amino acid selected from the group consisting of D87, D127, E94 and E125 of BxpB . In the foregoing embodiments, any donor sequence disclosed herein for BclA may be used. As shown in the examples, a variety of donor sequences may be used. In a specific embodiment, the donor sequence contains residue A20 of BclA. As discussed above, the acceptor sequence may contain a specified number of residues on the left and/or right (such as, but not limited to, at least 5, at least 10, at least 20 or at least 30 amino acid residues immediately left and/or right 5 or less, 10 or less, 20 or less or 30 or less amino acid residues immediately left and/or right) of the specified residue or be the full length polypeptide.

In another embodiment, the donor sequence is a an amino terminal sequence of the CotY polypeptide or the full length CotY polypeptide and the acceptor sequence is an amino acid sequence containing: (i) at least one amino acid selected from the group consisting of D5, D12, E7 and E14 of BxpB; (ii) at least one amino acid selected from the group consisting of D141 and E71 of CotY; (iii) at least one amino acid selected from the group consisting of D27, D89, E67 and E86 of ExsY; and/or (iv) at least one amino acid selected from the group consisting of D61, D69, D85, D93, D99, D100, E3, E27, E46, E55, E57, E75, E79, E86, E115, E136 and E140 of CotE. In one embodiment, the acceptor sequence contains at least one amino acid selected from the group consisting of D61 and D85 of CotE. As discussed above, such acceptor sequence may contain a specified number of residues on the left and/or right (such as, but not limited to, at least 5, at least 10, at least 20 or at least 30 amino acid residues immediately left and/or right 5 or less, 10 or less, 20 or less or 30 or less amino acid residues immediately left and/or right) of the specified residue or be the full length polypeptide. In the foregoing embodiments, any donor sequence disclosed herein for CotY may be used. As shown in the examples, a variety of donor sequences may be used. In a specific embodiment, the donor sequence contains residue S2 of CotY.

In another embodiment, the donor sequence is a an amino terminal sequence of the ExsY polypeptide or the full length ExsY polypeptide and the acceptor sequence is an amino acid sequence containing: (i) at least one amino acid selected from the group consisting of D5, D12, E7 and E14 of BxpB; (ii) at least one amino acid selected from the group consisting of D141, E7 and E71 of CotY; (iii) at least one amino acid selected from the group consisting of D17, D27, D89, E67 and E86 of ExsY; and/or (iv) at least one amino acid selected from the group consisting of D69, D99, D100, E6, E27, E31, E46, E55, E57, E75, E79, E86, E102, E115, E130, E136, E140 and E154 of CotE. In one embodiment, the acceptor sequence contains at least one amino acid selected from the group consisting of E6, E31, E102 or E154 of CotE. As discussed above, such acceptor sequence may contain a specified number of residues on the left and/or right (such as, but not limited to, at least 5, at least 10, at least 20 or at least 30 amino acid residues immediately left and/or right 5 or less, 10 or less, 20 or less or 30 or less amino acid residues immediately left and/or right) of the specified residue or be the full length polypeptide. In the foregoing embodiments, any donor sequence disclosed herein for ExsY may be used. As shown in the examples, a variety of donor sequences may be used. In a specific embodiment, the donor sequence contains residue S2 of ExsY.

In another embodiment, the donor sequence is a an amino terminal sequence of the ExsB polypeptide or the full length ExsB polypeptide and the acceptor sequence is an amino acid sequence containing: (i) at least one amino acid selected from the group consisting of D8, D13, D15, D95, D141, E7 and E90 of CotY; (ii) at least one amino acid selected from the group consisting of D17, D27, D137, E24 and E38 of ExsY; and/or (iii) at least one amino acid selected from the group consisting of D93, E27, E46, E55, E57, E79, E115 and E132 of CotE. In one embodiment, the acceptor sequence contains at least one amino acid selected from the group consisting of E132 of CotE, E24, E38 or D137 of ExsY or D8, D13, D15, D95 or E90 of CotY. As discussed above, such acceptor sequence may contain a specified number of residues on the left and/or right (such as, but not limited to, at least 5, at least 10, at least 20 or at least 30 amino acid residues immediately left and/or right 5 or less, 10 or less, 20 or less or 30 or less amino acid residues immediately left and/or right) of the specified residue or be the full length polypeptide. In the foregoing embodiments, any donor sequence disclosed herein for ExsB may be used. As shown in the examples, a variety of donor sequences may be used. In a specific embodiment, the donor sequence contains residue E18 of ExsB.

### Uses of Donor and Acceptor Sequences

The donor and acceptor sequences of the present disclosure have a number of uses. In one embodiment, the donor and acceptor sequences may be used to create a linkage between two targets. Targets include, but are not limited to, polypeptides. In another embodiment, the donor and acceptor sequences may be used in any application in which a binding pair, such as, but not limited to, an antibody and antigen or biotin and streptavidin/avidin, are used.

The reaction between the donor and acceptor sequences is capable of occurring over a broad range of conditions. For example, donor and acceptor sequences are capable of forming covalent bonds over a broad temperature range. Reactions between polypeptides containing donor and acceptor sequences to form covalent bonds have been successful at room temperature as well as in incubations on ice and at temperatures over 88 degrees F. Reactions between polypeptides containing donor and acceptor sequences to form covalent bonds have been successful when conducted in a buffer containing high concentrations of SDS and dithiothreitol (DTT). Furthermore, the reaction between the donor and acceptor sequences is rapid occurring in as little as 30 seconds or less.

As a result, the donor and acceptor sequences of the present disclosure may be used to create linkages between targets under a broad range of conditions in which other biding pairs are not operative.

### Use as Immunogens

The donor and acceptor sequences of the present disclosure may be used to create an immunogen for use in creating vaccines and the like. In one embodiment, the immunogen comprises a backbone sequence containing one or more acceptor sequences to which an antigenic agent, such as an antigenic polypeptide, containing a donor sequence can bind.

In one embodiment, the backbone sequence is as a full length BxpB, CotE, CotY or ExsY polypeptide. In addition, multiple copies of such full length polypeptides (in any combination) may be created by linking the sequences together directly or through a linking sequence. Furthermore, one or more full length sequences may be combined with acceptor sequences that are fragments of the full length sequences. In another embodiment, the backbone sequence is a fragment of a BxpB, CotE, CotY or ExsY polypeptide; such fragments may be 10, 20, 30, 40, 50, 75 or 100 amino acids in length or greater. In still another embodiment, the backbone sequence is a polypeptide sequence not otherwise associated in nature with a sequence from a BxpB, CotE, CotY or ExsY polypeptide, said polypeptide sequence containing one or more acceptor sequences. In the foregoing, the backbone sequence may contain 1, 5, 10, 15, 20, 25 or more acidic residues. In one embodiment, the backbone sequence contains 10-25 or more acidic residues.

In a specific embodiment, the backbone is a full length BxpB polypeptide or multiple copies of the full length BxpB polypeptide linked together, directly or via linking sequence. In another specific embodiment, the backbone is a full length BxpB polypeptide or multiple copies of the full length BxpB polypeptide containing one or more acceptor sequences from a BxpB, CotE, CotY or ExsY polypeptide. In a specific embodiment, such acceptor sequences are from BxpB; such sequences include sequences containing one or more of amino acid residues selected from the group consisting of D87, E94, E125 and D127.

The donor sequence may be any donor sequence disclosed herein. In a specific embodiment, the donor sequence is a fragment of the NTD of the ExsB, BclA, CotY and ExsY polypeptides. In another embodiment, the donor sequence is a donor sequence described from the BclA polypeptide. In a further embodiment, the donor sequence is amino acids 1-40, 1-38, 1 and 20-38, 20-33, 20-38, 10-35 or 20-35 of BclA.

The donor sequences in a particular immunogen as described may be the same or may be different. In other words, various antigenic agents may be combined with various donor sequences as disclosed herein.

The nature of the antigenic agent determines the specificity of the immune response directed by the immunogen. The antigenic agent may be any antigenic agent known in the art and may be coupled with a given donor sequence as is known in the art and described herein. In a specific embodiment, the antigenic agent is from a Bacillus species, such as, B. anthracis, Bacillus thuringiensis or Bacillus cereus. In one embodiment, the antigenic agent is from B. anthracis. Antigens from Bacillus species are known in the art and are described in WO/2008/048344. Representative antigens include, but are not limited to, protective antigen, lethal factor and edema factor.

The immunogen described may contain a single type of antigenic agent (preferably multiple copies) or may contain more than one type of antigenic agent. For example, an immunogen for use in a vaccine against B. anthracis may contain only protective antigen or protective antigen in combination with edema factor and/or lethal factor.

### Use in Purification Strategies

The donor and acceptor sequences of the present disclosure may be used for purification of a desired polypeptide or other target. For simplicity, the discussion below will refer to polypeptides only. In one embodiment, the DNA sequence specifying a donor or acceptor sequence of the present disclosure is attached to a polypeptide of interest, either directly or through the use of a linker sequence. Alternatively, an isolated donor or acceptor sequence may be linked chemically or through other means to the polypeptide. Still further, the polypeptide may be produced by recombinant means and designed to incorporate a donor or acceptor sequence. In one embodiment of the foregoing, a linker sequence is used. When used, the linker sequence may contain a restriction site or other site to allow the donor or acceptor sequence to be cleaved from the polypeptide of interest. Techniques for attaching a donor or acceptor sequence to a protein of interest are well known in the art. The polypeptide of interest with the attached donor or acceptor sequence is then expressed. The polypeptide of interest with the attached donor or acceptor sequence is then reacted with a composition comprising the other of the donor or acceptor sequence (for example, if the polypeptide of interest contains the donor sequence, it is reacted with a composition comprising an acceptor sequence and vice versa). The donor and acceptor sequences form a covalent bond, thereby purifying the polypeptide of interest.

In a specific embodiment, the polypeptide of interest is linked to a donor sequence, either directly or through a linker as discussed above. In this embodiment, the donor sequence may be any donor sequence disclosed herein. In one embodiment, the donor sequence is from the BclA, ExsB, ExsY or CotY polypeptides. In a specific embodiment, the donor sequence is from the BclA polypeptide. In any of the foregoing, the donor sequence may be a fragment of the above-referenced polypeptides, such as a 5, 10, 15, 20, 25, 30, 35 or 40 amino acid fragments from the NTD of the referenced polypeptides. In a specific embodiment, the donor sequence is an amino acid sequence specified for BclA as described herein. The acceptor sequence may be any acceptor sequence disclosed herein. In one embodiment, the acceptor sequence is a full length polypeptide, such as a full length BxpB, CotE, CotY or ExsY polypeptide. In another embodiment, the acceptor sequence is a full length BxpB polypeptide. In another embodiment, the acceptor sequence is a fragment of a BxpB, CotE, CotY or ExsY polypeptide; such fragments may be 10, 20, 30, 40, 50, 75 or 100 amino acids in length or greater. The acceptor sequence may be immobilized such as on a column and the polypeptide of interest containing the donor sequence purified through column chromatography as is known in the art. Alternatively, the acceptor sequence may be attached to a plate or dish, such as a microtiter plate as well.

### Use in Detection

The donor and acceptor sequences of the present disclosure may be used for detection of a target. In one aspect of such a use, a polypeptide expressing a donor or acceptor sequence of the present disclosure is separated by gel electrophoresis or other means known in the art. A polypeptide containing the other of the donor or acceptor sequence may be used to bind to the donor or acceptor sequence on the polypeptide to be detected. In such an embodiment, the donor and acceptor sequences may be used in place of antibody based detection techniques.

### Modified Polypeptides

The present disclosure also provides for modified polypeptides consisting of, consisting essentially of or comprising a donor sequence as disclosed herein. The present disclosure further provides for modified polypeptides consisting of, consisting essentially of or comprising an acceptor sequence as disclosed herein.

For example, embodiments of the present disclosure provide a donor fusion protein comprising a donor polypeptide sequence linked to a second polypeptide. In one embodiment, the donor polypeptide sequence is a polypeptide sequence from a BclA, CotY, ExsY or ExsB polypeptide; donor sequences from one or more of the foregoing proteins may be included. Any donor sequence disclosed herein may be used in such a donor fusion protein. In one embodiment, the donor sequence is a full length BclA, CotY, ExsY or ExsB polypeptide. In another embodiment, the donor sequence is a fragment of a full length BclA, CotY, ExsY or ExsB polypeptide. In yet another embodiment, the donor sequence is a fragment of a full length BclA, CotY, ExsY or ExsB polypeptide selected from the group consisting of: the first 40 amino acid residues, the first 38 amino acid residues, the first 20 amino acid residues, the first 10 amino acid residues, amino acid residues 2-40, amino acid residues 2-38, amino acid residues 20-38, amino acid residues 1 and 20-38, amino acid residues 2-38 of the foregoing polypeptides.

In one embodiment, the second polypeptide of the donor fusion protein is taken from a polypeptide that is different from the polypeptide from which the donor sequence is derived. In another embodiment, the second polypeptide of the donor fusion protein is taken from a non-BclA, -CotY, -ExsY and -ExsB polypeptide.

In addition, embodiments of the present disclosure provide an acceptor fusion protein comprising an acceptor polypeptide sequence linked to a second polypeptide. In one embodiment, the acceptor polypeptide sequence is a polypeptide sequence from a BxpB, CotE, CotY or ExsY polypeptide; acceptor sequences from one or more of the foregoing proteins may be included. Any acceptor sequence disclosed herein may be used in such an acceptor fusion protein. In one embodiment, the acceptor sequence is a full length BxpB, CotE, CotY or ExsY polypeptide. In another embodiment, the acceptor sequence is a fragment of a full length BxpB, CotE, CotY or ExsY polypeptide. In yet another embodiment, the acceptor sequence is a fragment of a full length BxpB, CotE, CotY or ExsY polypeptide selected from the group consisting of: a fragment at least 25 amino acids in length containing one or more acidic residues, a fragment at least 50 amino acids in length containing one or more acidic residues, a fragment at least 75 amino acids in length containing one or more acidic residues, a fragment at least 100 amino acids in length containing one or more acidic residues, a fragment at least 125 amino acids in length containing one or more acidic residues or a fragment at least 150 amino acids in length containing one or more acidic residues. In the foregoing, in one embodiment, such fragment contains 2, 3, 4, 5, 6, 7, 8, 9, 10 or more acidic residues.

In one embodiment, the second polypeptide of the acceptor fusion protein is taken from a polypeptide that is different from the polypeptide from which the acceptor sequence is derived. In another embodiment, the second polypeptide of the acceptor fusion protein is taken from a non-BxpB, -CotE, -CotY or -ExsY polypeptide.

In one embodiment, the second polypeptide is an antigenic agent. The antigenic agent may be any antigenic agent known in the art and may be coupled with a given donor sequence as is known in the art and described herein. In a specific embodiment, the antigenic agent is from a Bacillus species, such as, B. anthracis, B. thuringiensis or B. cereus. In one embodiment, the antigenic agent is from B. anthracis. Antigens from Bacillus species are known in the art and are described in WO/2008/048344. Representative antigens include, but are not limited to, protective antigen, lethal factor and edema factor.

In another embodiment, the second polypeptide is an antibody or antibody fragment. As referred to herein, an antibody fragment may include any suitable antigen-binding antibody fragment known in the art as well as heavy chain or a portion (i.e., fragment) thereof. The antibody fragment may be obtained by manipulation of a naturally-occurring antibody, or may be obtained using recombinant methods. For example, the antigen-binding antibody fragment may include, but is not limited to Fv, single-chain Fv (scFV; a molecule consisting V_{L} and V_{H} connected with a peptide linker), Fab, Fab₂, single domain antibody (sdAb), and multivalent presentations of the foregoing. The antigen-binding antibody fragment may be derived from any one of the known heavy chain isotypes: IgG, IgM, IgD, IgE, or IgA. In one embodiment, the antibody fragment may comprise an immunoglobulin heavy chain or a portion (i.e., fragment) thereof. For example, the heavy chain fragment may comprise a polypeptide derived from the Fc fragment of an immunoglobulin, wherein the Fc fragment comprises the heavy chain hinge polypeptide, and C_{H2} and C_{H3} domains of the immunoglobulin heavy chain as a monomer. The heavy chain (or portion thereof) may be derived from any one of the known heavy chain isotypes: IgG, IgM, IgD, IgE, or IgA. In addition, the heavy chain (or portion thereof) may be derived from any one of the known heavy chain subtypes: IgG1, IgG, IgG3, IgG4, IgA1 or IgA2.

In one embodiment, the fusion proteins above comprises an interdomain linker linked to a donor or acceptor sequence such that the one end of the donor or acceptor sequence is linked to one end of the interdomain linker and the other end of the interdomain linker is linked to the second polypeptide.

### EXAMPLES

### Example 1

To test and clarify the model that the amino terminus of cleaved BclA is covalently attached to BxpB, purified exosporia from spores of the B. anthracis Sterne strain were prepared. The Sterne stain is avirulent due to its inability to produce a capsule on vegetative cells; however, the exosporium of Sterne spores is essentially identical to the exosporium produced by virulent B. anthracis stains (14). The purified exosporia were incubated under denaturing and reducing conditions to solubilize exosporium proteins and proteins complexes, which were separated by SDS-PAGE. The >250-kDa complexes containing BclA and BxpB were excised from the gel and treated in situ with trypsin and chymotrypsin (15). Trypsin and chymotrypsin cleave BxpB at many sites but only chymotrypsin cleaves the NTD of BclA; one of the chymotrypsin cleavage sites of the NTD is between residues F21 and D22. Therefore, according to the model disclosed herein, trypsin and chymotrypsin treatment of BclA-BxpB covalent complexes should produce peptides with the BclA dipeptide containing residues A20 and F21 (AF peptide) linked to an amino acid within a proteolytic fragment of BxpB. To identify these peptides, the proteolytic fragments of the >250-kDa complexes were separated by liquid chromatography, and the major fragments were sequenced by tandem mass spectrometry (LC-MS/MS). The attachment of an AF peptide to a particular amino acid was detected as an increase of 218.1 Da in the expected mass of that amino acid.

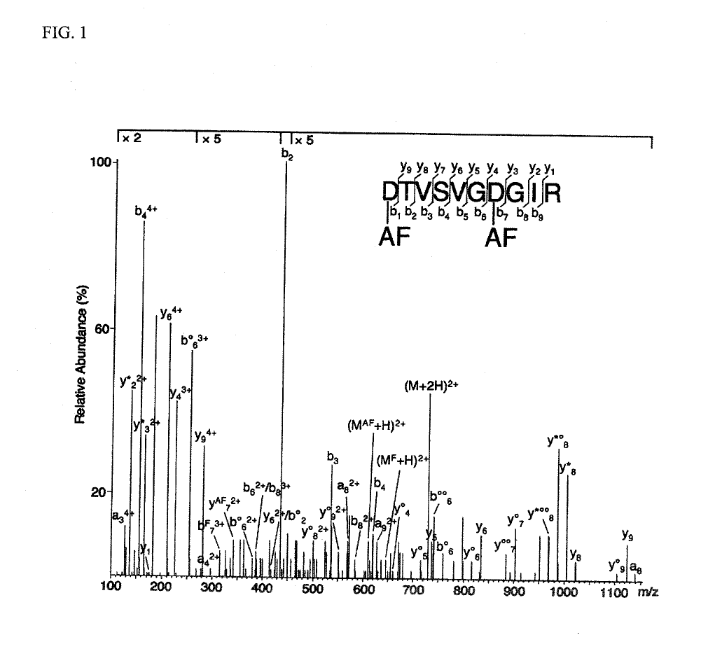

Many proteolytic fragments containing only BclA, BxpB, ExsY, or CotY sequences were identified. In addition, eight BxpB fragments with one or two attached AF peptides were identified (Table 1). The MS/MS spectrum of one of these fragments is shown in FIG. 1. In each of the eight compound fragments, the AF peptide was attached to an internal acidic (D or E) residue of BxpB, which was accompanied by the loss of mass of one water molecule. This result indicated the formation of an isopeptide bond between the amino group of BclA residue A20 and a side-chain carboxyl group of BxpB. The attachment of an AF peptide occurred at eight of the 13 acidic residues of BxpB, which contains 167 amino acids (9). Comparing independently-derived fragments containing the same BxpB residues showed that a particular acidic residue might be involved in an isopeptide bond in one fragment but not in another (Table 1), indicating a somewhat random pattern of AF peptide attachment. On the other hand, none of the acidic residues near the amino terminus of BxpB (i.e., D5, E7, D12, and E14) participated in the formation of an isopeptide bond with BclA. These results demonstrate that BclA is attached to BxpB through formation of isopeptide bonds.

### Example 2

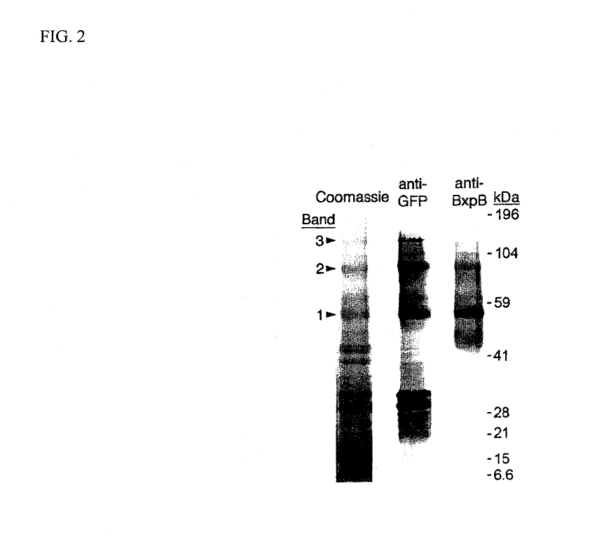

To further investigate the mechanism of BclA attachment to BxpB, plasmid-encoded BclA NTD-enhanced green fluorescence proteins (eGFP) fusion protein were expressed in BclA-deficient B. anthracis strain CLT360 (ΔbclA ΔrmlD)/pCLT1525 (13). The ΔrmlD mutation in this strain prevents rhamnose biosynthesis and stabilizes the fusion protein on the spore surface for unknown reasons. The BclA NTD directs stable attachment of the fusion protein to the exosporium basal layer of spores produced by this strain (12, 13). Exosporia were purified from these spores, exosporium protein complexes were separated by SDS-PAGE as described above in duplicate gels, and protein bands in the gels were analyzed by immunoblotting with either an anti-BxpB MAb (13) or a commercially available anti-eGFP MAb. Three major eGFP-containing protein bands with apparent molecular masses large enough to contain fusion protein-BxpB complexes, which have a minimum calculated molecular mass of 46.5 kDa, were detected. These protein bands had apparent molecular masses of 55, 90, and 130 kDa and were designated bands 1, 2, and 3, respectively (FIG. 2). The relative levels of anti-eGFP MAb staining of these three bands was 1>2>>3. Using densitometry, the intensities of staining of each band with the anti-BxpB and eGFP MAbs were measure and the relative amounts of BxpB and eGFP in each band were calculated. These results indicated that bands 1, 2, and 3 contained one, two, and three fusion proteins per molecule of BxpB, respectively. Based on their apparent molecular masses, and assuming slightly slower gel mobility due to a branched protein structure, the results show that the complexes in bands 1, 2, and 3 contain a single molecule of BxpB.

To substantiate these conclusions, protein bands 1 and 2 were individually digested with trypsin and chymotrypsin, and the resulting peptides were separated and sequenced by LC-MS/MS as described above. Eighteen BxpB fragments with attached AF peptides derived from the BclA NTD-eGFP fusion protein were identified (Table 2). Sixteen fragments—seven from band 1 and nine from band 2—contained a single AF peptide. The remaining two fragments contained two AF peptides, and both of these fragments were obtained from band 2. These results are consistent with the prediction that bands 1 and 2 contain BxpB-(BclA NTD-eGFP) and BxpB-(BclA NTD-eGFP)₂ complexes, respectively. Furthermore, the analysis of the fragments from bands 1 and 2 showed that the attachment of AF peptides occurred at eight different BxpB residues, six acidic residues identified in Table 1 along with residues E7 and D141.

Taken together, the results of the analyses of fragments derived from both BxpB-BclA and BxpB-(BclA NTD-eGFP) complexes indicate that up to three BclA NTDs can be attached through isopeptide bonds to a single molecule of BxpB. However, attachment of multiple NTDs to a single BxpB proteolytic fragment containing at least two acidic residues was much more frequent when the NTD was derived from BclA. The frequency of multiple attachments was 57% with BclA compared to 18% with BclA NTD-eGFP (considering only fragments derived from band 2). This difference might be due to the fact that BclA is attached as a trimer while the fusion protein is presumably attached as a monomer. The covalent attachment of one strand of the BclA trimer to BxpB could facilitate attachment of the second and third strands of this trimer to nearby BxpB acidic residues. Such a model is consistent with the observation that multiple BclA NTDs are readily attached to neighboring BxpB acidic residues (Table 1) and with the fact that less than 10% of the BclA extracted from spores is monomeric (13).

The results shown in Tables 1 and 2 demonstrate that BclA NTD attachment can occur at 10 of the 13 widely scattered acidic residues of BxpB. Attachment to the BxpB amino-terminal residues D5, D12, and E14 was not detected, although numerous BxpB fragments including these residues were identified by LC-MS/MS.

### Example 3

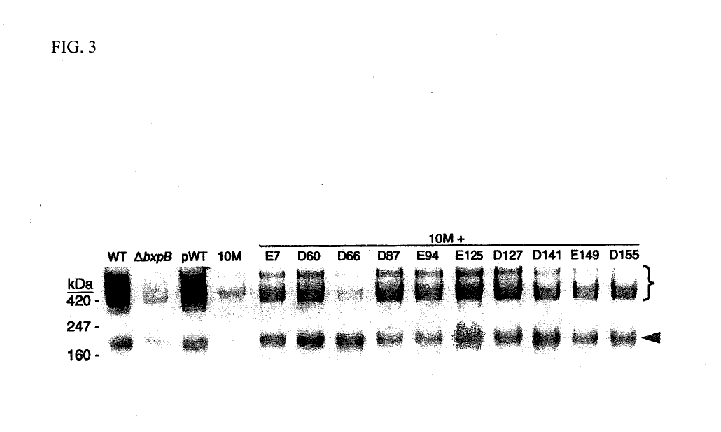

To further investigate the selection of BclA attachment sites, a series of plasmids capable of expressing, from the bxpB promoter, wild-type BxpB and BxpB mutants in which selected acidic residues were changed to alanines, were constructed. The mutations included changing all 13 acidic residues (designated 13M), changing all acidic residues except D5, D12, and E14 (designated 10M), and changing all acidic residues except D5, D12, E14, and one of the other 10 D/E residues (designated 10M+the other retained D/E residue). The expression plasmids were individually introduced by transformation into a ΔbxpB variant of the Sterne strain (CLT307), and formation of >250-kDa complexes containing BclA and BxpB was examined during sporulation. These complexes were detected by immunoblotting with an anti-BclA MAb (FIG. 3), and the presence of wild-type or mutant BxpB proteins was confirmed by immunoblotting with an anti-BxpB MAb (data not shown) (13) or by MS/MS analysis of proteolytic fragments as described above, respectively.

In the case of the 13M and 10M mutants, only background levels of >250-kDa complexes equal to that observed with a ΔbxpB variant of the Sterne strain were detected (FIG. 3 and data not shown). Presumably, this background was due to low-level BclA attachment to the BxpB paralog ExsFB. The failure to detect BclA attachment to the 10M mutant, which did not appear to be due to mutant protein instability (see below), provided direct evidence that BxpB residues D5, D12, and E14 cannot participate in BclA attachment. In contrast, >250-kDa complexes above background levels were detected when every other mutant BxpB was expressed (FIG. 3), confirming that all BxpB D/E residues other than D5, D12, and E14 are potential sites for BclA attachment. However, the level of BclA attachment to individual D/E residues was highly variable, suggesting preferred sites. The highest levels of attachment were observed at residues E125 and D127, which were approximately one-third that of the level observed with wild-type BxpB (FIG. 3). To confirm that attachment of BclA to the 10M+D/E mutant proteins occurred through isopeptide bonds, we analyzed >250-kDa complexes formed by the 10+E125 mutant by LC-MS/MS as described above. A branched peptide in which an AF peptide was cross-linked to residue E125 was identified. Furthermore, several branched peptides in which an AF peptide derived from the BclA NTD-eGFP fusion protein was cross-linked to residue E125 of the 10M+E125 mutant BxpB were detected (data not shown).

### Example 4

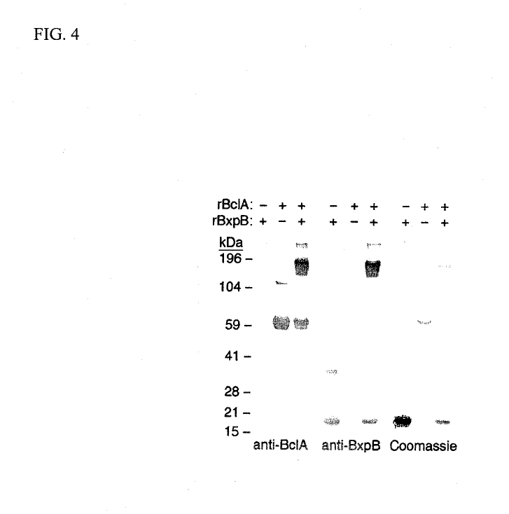

To examine the possibility that BclA and BxpB form isopeptide bonds without the participation of other proteins, amino-terminal His₆-tagged versions of BclA and BxpB in Escherichia coli were constructed and each recombinant (r) protein purified by affinity chromatography (9, 10). The His₆ tag was removed from rBxpB (10). The two proteins were combined at μM concentrations in phosphate buffered saline and incubated at room temperature for 30 min. After separation by SDS-PAGE, stable and high-molecular-mass complexes containing both rBclA and rBxpB were detected by immunoblotting individually with anti-BclA and anti-BxpB MAbs and by staining with Coomassie Blue (FIG. 4). These complexes were excised from a polyacrylamide gel and treated in situ with trypsin and chymotrypsin, and the proteolytic fragments were analyzed by LC-MS/MS as described above. A total of 32 branched peptides were identified in which a peptide derived from the amino-terminal region of rBclA (either GSSHHHHHHSSGL or GSSHHHHHHSSGLVPR; residues 2-14 or 2-17, respectively) was attached to one or two internal acidic residues of a proteolytic fragment of rBxpB (Table 3). Again, this attachment was accompanied by the loss of mass of one water molecule, consistent with isopeptide bond formation. In these branched peptides, isopeptide bonds were formed between the amino group of rBclA residue G2 and the side-chain carboxyl groups of any of the 13 acidic residues of rBxpB. Presumably, the initiating methionine residue of rBclA was removed by a methionylaminopeptidase in E. coli. The above results show that BclA-BxpB isopeptide bonds form spontaneously in vitro.

In the analysis of isopeptide bond formation in vivo and in vitro, samples were heated at 100° C. prior to SDS-PAGE. Control experiments were performed demonstrating that the same isopeptide bonds were formed without heating (data not shown).

### Example 5

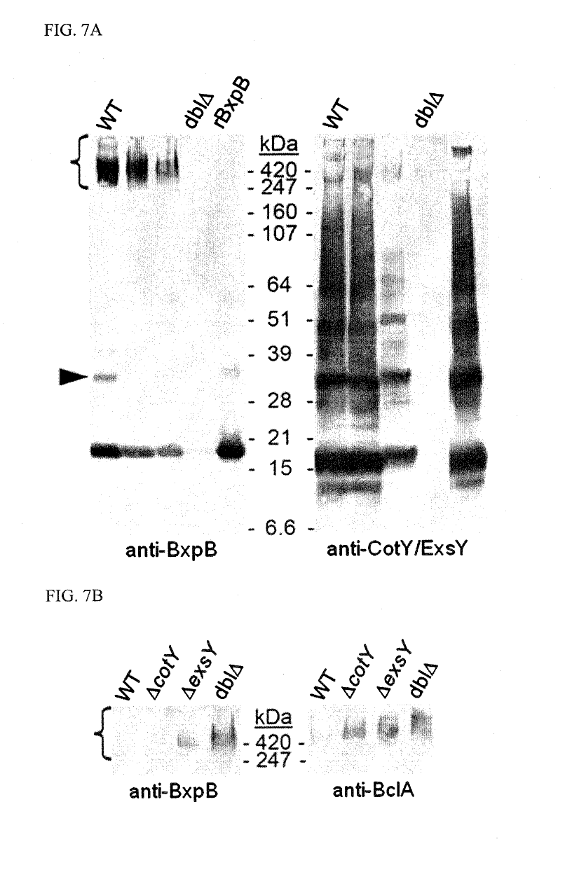

The B. anthracis exosporium contains stable high-molecular-mass (>250-kDa) complexes that include BclA, BxpB, ExsY, and/or CotY (13). To further examine these protein complexes, exosporium proteins were extracted by boiling purified spores of B. anthracis wild-type (WT) strain or its variants (ΔcotY, ΔexsY, ΔcotY/ΔexsY and ΔbxpB) in sample buffer containing 4% SDS and 100 mM DTT. Solubilized proteins and protein complexes were separated by SDS-PAGE and analyzed by immunoblotting with anti-BxpB or anti-ExsY/CotY MAbs, respectively (FIG. 7A). The anti-BxpB MAb does not react with the BxpB paralog ExsFB (9), and the anti-ExsY/CotY MAb reacts similarly with ExsY and CotY (32). As expected, >250-kDa complexes that reacted with both the anti-BxpB MAb and the anti-ExsY/CotY MAb were detected (FIG. 7A, lane 1-3). Free monomeric BxpB, ExsY, or CotY, which have molecular masses of 17.3, 16.1, and 16.8 kDa, respectively were also detected. Interestingly, multiple ladder-like major bands were detected in the WT, ΔcotY, ΔexsY, and ΔbxpB spores, with apparent molecular masses corresponding to the dimer, trimer, tetramer, and pentamer of ExsY and/or CotY, respectively (FIG. 7A, lane 1-3, and 6). These bands are stable in the presence of a high level of SDS and DTT, suggesting the presence of a stable linkage, other than a disulfide bond, between a dimer of ExsY and/or CotY. Furthermore, a BxpB-containing band with an apparent molecular mass of 33 kDa, which is smaller than a BxpB dimer, appeared to also contain ExsY, but not CotY (compare lane 1 with lane 2, 3 and 5 in FIG. 7A). These results showed that BxpB and ExsY as well as ExsY and/or CotY multimers could be cross-linked by a stable, perhaps covalent, linkage other than a disulfide bond.

The ΔcotY spores have an apparently intact exosporium like the WT spores (data not shown) whereas the ΔexsY spores only retain a cap-like exosporium fragment covering about one quarter of spore surface when grown on solid medium (32). The ΔexsYΔcotY double-mutant spores lack exosporium when grown on solid medium (FIG. 7A, lane 4 and data not shown), indicating that both ExsY and CotY are required for the exosporium assembly of B. anthracis, consistent with similar conclusions in B. cereus (33). To further investigate whether BclA and BxpB are incorporated into high-molecular-mass complexes in the absence of ExsY and/or CotY, we isolated and concentrated the supernatant of the spore cultures and analyzed it by SDS-PAGE as described above, followed by the immunoblotting with anti-BxpB, anti-BclA, and anti-ExsY/CotY MAbs, respectively (13). Interestingly, the amount of high-molecular-mass (>250-kDa) complexes containing BclA and BxpB in the supernatant of WT, ΔcotY, ΔexsY, and ΔexsYΔcotY spore cultures were gradually increased (FIG. 7B), and these complexes did not react with the anti-ExsY/CotY MAb (data not shown). In contrast, the amount of >250-kDa complexes detected in the WT, ΔcotY, ΔexsY, and ΔexsYΔcotY spores were gradually reduced to an undetectable level (FIG. 7A, lane 1-4). These results demonstrated that, even in the absence of ExsY and CotY, BclA and BxpB still are incorporated into the >250-kDa complexes, the assembly of which, however, requires ExsY and/or CotY, with ExsY playing a dominant role.

### Example 6

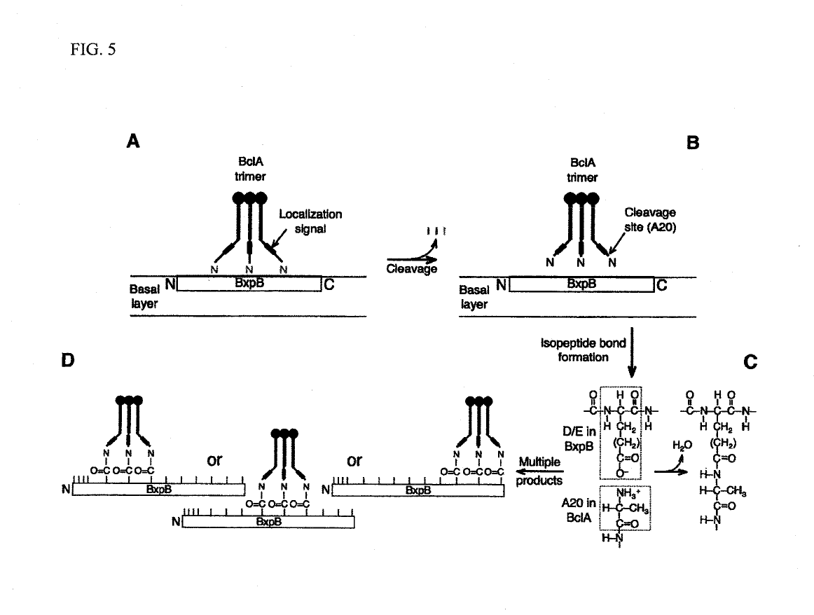

In addition to the isopeptide bond formation between BclA and BxpB, isopeptide bond formation was also demonstrated between the B. anthracis exosporium proteins CotY, ExsY, ExsB, BxpB, and CotE. Table 4 shows the isopeptide bond formation formed in vivo as determined by the methods described above. The results show that the ExsB polypeptide functions as a donor only, the BxpB and CotE polypeptides function as acceptors only, while the CotY and ExsY polypeptides function as both donors and acceptors. Table 4 shows that CotY and ExsY are capable of forming isopeptide bonds with BxpB, CotY, ExsY and CotE, and that ExsB is capable of forming isopeptide bonds with CotY, ExsY and CotE. The amino acid residues involved in isopeptide bond formation are specified in Table 4. It is noted that BclA does not form isopeptide bonds with CotY, ExsY or CotE and that ExsB does not form isopeptide bonds with BxpB. In addition, CotY and ExsY only form isopeptide bonds with acidic residues in the first 14 amino acids of BxpB (D5, D12, E7 and E14). As discussed above, BclA did not form isopeptide bonds in vivo with residues D5, D12 or E14. Still further it is noted that no isopeptide bonds were found involving the last 26 residues of CotE, which contains 14 acidic residues, suggesting these residues are not available for binding. A model of isopeptide bond formation in the exosporium of B. anthracis is shown in FIG. 5.

### Example 7

The data in FIG. 7 suggested that BxpB and ExsY are cross-linked by a stable linkage other than a disulfide bond. Since BclA is attached to BxpB via the formation of isopeptide bonds between the proteolytically processed BclA residue A20 and a side chain of an acidic residue of BxpB (see above), and the initiating methionine residues of both ExsY and CotY were removed to provide an amino terminus of S2 presumably by a methionyl-aminopeptidase in B. anthracis, analogous mechanism of isopeptide bond formation could be involved in the cross-linking of proteolytically processed amino terminus (residue S2) of ExsY and/or CotY to BxpB. To test this possibility, exosporia from spores of the B. anthracis Sterne strain were purified and exosporium proteins and protein complexes separated by SDS-PAGE. The >250-kDa complexes containing BclA, BxpB, ExsY and/or CotY were then excised from the gel and treated in situ with trypsin and chymotrypsin (15). Trypsin and chymotrypsin cleave BxpB, ExsY, and CotY at many sites including those in their amino terminal sequences. As the starting sequences of mature ExsY and CotY are SCNENK and SCNCN, respectively, and considering the possible (and frequent) missing cleavages after N residues by chymotrypsin, the double digestions of trypsin and chymotrypsin of these complexes will potentially produce three peptides (SCN, SCNEN, and SCNENK) from ExsY or two peptides (SCN and SCNCN) from CotY. These peptides (designated cross-linkers or amino terminal fragments) are shown to foam a linkage to a side chain of a D/E residue within BxpB. Since there are no more cleavage sites of trypsin or chymotrypsin in the next nine residues of ExsY or CotY, no more cross-linkers were considered. To identify these branched fragments, the proteolytic fragments of the >250-kDa complexes were analyzed by LC-MS/MS as described herein. The attachment of an amino terminal fragment to a particular D/E residue was detected as an increase of the calculated mass of the fragment (e.g., 361.1 Da for SCN in which the C residue was modified by carbamidomethylation) in the expected mass of the D/E residue.

Many proteolytic fragments containing only BclA, BxpB, ExsY, or CotY (also ExsB, see below) sequences were identified. In addition, 12 BxpB fragments with one or two cross-linkers from ExsY and/or CotY were identified (Table 5). In each of the branched fragments, an amino terminal fragment was attached to an internal D/E residue of BxpB, which was accompanied by the loss of mass of one water molecule. This result demonstrated the formation of an isopeptide bond between the free amino group of residue S2 of ExsY/CotY and a side-chain carboxyl group of a D/E residue of BxpB. Comparing independently-derived fragments containing the same BxpB residues showed that a particular acidic residue might be involved in an isopeptide bond in one fragment but not in another (Table 5), consistent with a somewhat random pattern attachment as described for BclA attachment to BxpB. Interestingly, only the amino-terminal BxpB residues D5, E7, D12, and E14 participated in isopeptide bond formation with ExsY/CotY (FIG. 8). Notably, these were the BxpB acidic residues that were not found to participate in isopeptide bond formation with BclA in vivo (note these residues were found to participate in isopeptide bond formation with BclA in vitro).

### Example 8

Similar to the ExsY/CotY attachment to BxpB, ExsY/CotY could also be cross-linked to another ExsY/CotY by isopeptide bonds. As ExsY and CotY contain 15 and 18 acidic residues, respectively (FIG. 9), it is possible that ExsY and/or CotY form isopeptide bonds with one another through an analogous mechanism of isopeptide bond formation as described above. By further analyzing the LC-MS/MS data described above, nine branched peptides were identified in which one or two fragments derived from the amino-terminal region of ExsY/CotY were attached to one or two internal acidic residues of a proteolytic fragment of ExsY/CotY (Table 6). Again, this attachment was accompanied by the loss of mass of one water molecule, consistent with isopeptide bond formation. Interestingly, isopeptide bonds could be formed between two molecules of the same protein (ExsY or CotY). 5 of 15 acidic residues of ExsY, as well as 3 of 18 acidic residues of CotY, were observed to participate in isopeptide bonds. Most (6 of 8) of the cross-linking sites were shared with both ExsY and CotY, however, D17 of ExsY and E7 of CotY were only occupied by ExsY (also ExsB, see below) (FIG. 9).

### Example 9

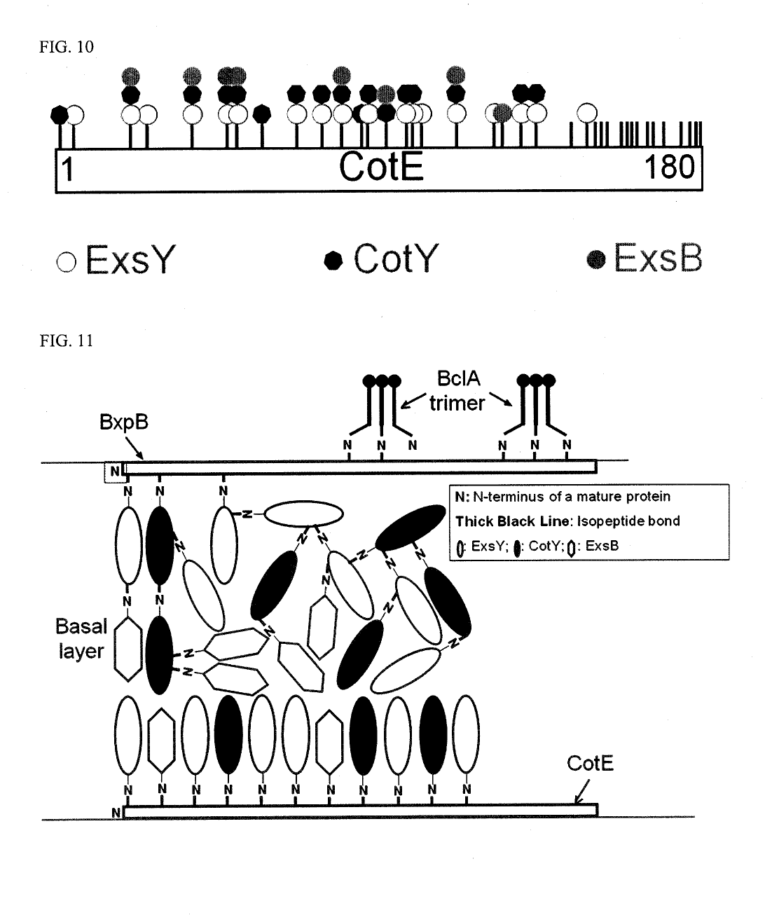

Like the ΔexsYΔcotY double-mutant spores, ΔcotE spores of B. anthracis also lack exosporium (34). CotE is a conserved morphogenetic protein in both B. anthracis and Bacillus subtilis with the latter, however, lacking the exosporium structure (34). In B. subtilis, CotE resides between the inner coat and outer coat layers in mature spore (35), and is essential for outer coat assembly. In B. anthracis, CotE is required for exosporium assembly and also has a modest role in coat protein assembly, suggesting that it might participate in connecting the exosporium to the coat surface. Furthermore, CotE is also incorporated into stable high-molecular-mass (>170-kDa) complexes at a late stage of sporulation (34). These raise the possibility that CotE directs exosporium assembly at least partially through the interactions, perhaps cross-links, with ExsY and/or CotY. To test this possibility, we further analyzed the LC-MS/MS data described above to search for branched peptides in which one or more fragments derived from the amino-terminal region of ExsY/CotY were attached to one or more internal acidic residues of a proteolytic fragment of CotE. Surprisingly, 45 such branched peptides were identified, indicating that at least three molecules of ExsY and/or CotY can be cross-linked to a single molecule of CotE via isopeptide bonds (Table 7). A total of 22 of 38 CotE acidic residues were found to participate in isopeptide bond formation with ExsY/CotY. 18 and 17 of the 22 cross-linking sites were occupied by ExsY and CotY, respectively, with 13 of them shared with both ExsY and CotY. No obvious selectivity was observed with these cross-links. However, no isopeptide bonds involving the 14 acidic residues located within the last 26 residues of CotE (i.e., residues 155-180) were observed in vivo (FIG. 10).

### Example 10

In the LC-MS/MS analysis of >250-kDa exosporium protein complexes described above, multiple proteolytic fragments of ExsB were also observed, some of which contain phosphorylated threonine residues as described previously (29). ExsB is a highly phosphorylated protein required for the stable attachment of the exosporium of B. anthracis (29). In B. subtilis, the assembly of an outer coat protein CotG, an ExsB orthologue, requires CotE (36). Similar to BclA, the amino terminus of ExsB is proteolytically processed to remove first 17 amino acids, leaving E18 as the new amino-terminal residue of the mature ExsB (37). All of these results raise the possibility that ExsB plays an important role in exosporium assembly, perhaps through formation of isopeptide bonds between the proteolytically processed amino terminus (residue E18) of ExsB and a side chain of an acidic residue of an acceptor protein (i.e., CotY, ExsY, or CotE). As the starting sequence of the mature ExsB is EDF, trypsin and chymotrypsin treatment of the >250-kDa complexes should produce peptides with the ExsB tripeptide (EDF peptide) linked to a side chain of an acidic residue within a proteolytic fragment of an acceptor protein. Therefore, the attachment of an EDF peptide to a particular D/E residue was detected as an increase of 391.1 Da in the expected mass of the D/E residue by the LC-MS/MS analysis.

As expected, a total of 18 branched peptides were identified in which one or two EDF peptides were attached to one or two internal acidic residues of a proteolytic fragment of ExsY, CotY, or CotE, but not BxpB (Tables 8-10 and data not shown). When ExsY was the acceptor protein, 5 of 15 acidic residues of ExsY were observed to participate in isopeptide bonds with ExsB. Of the five cross-linking sites, D17 was shared with ExsY; D27 was shared with both ExsY and CotY; and the other three sites were only occupied by ExsB (FIG. 9). When CotY was the acceptor protein, 7 of 18 acidic residues of CotY were observed to participate in isopeptide bonds with ExsB. D141 was shared with both ExsY and CotY, E7 was shared with ExsY, while the other five cross-linking sites were reserved only for ExsB (FIG. 9). When CotE was the acceptor protein, 8 of 38 acidic residues of CotE were observed to participate in isopeptide bonds with ExsB. D132 was a unique cross-linking site for ExsB whereas the other seven sites, except D93 only shared with CotY, were shared with both ExsY and CotY. Again, no isopeptide bonds involving the 14 acidic residues located within the residues 155-180 of CotE were observed (FIG. 10).

### Discussion

The results presented in this study demonstrate that this unusual mechanism of isopeptide bond formation is a conserved feature of exosporium assembly in B. anthracis. The present disclosure reveals a complicated exosporium protein network in which basal layer proteins BxpB, ExsY, CotY, ExsB, and CotE are also connected—or interconnected in this case—through isopeptide bonds. Even though there are no apparent similarities in the sequences of these proteins (except for homologous ExsY and CotY), the mechanisms for basal layer protein cross-linking appear to be analogous. First, the proteolytic cleavage of ExsY/CotY between residues M1 and S2 and the cleavage of ExsB between residues M17 and E18 generate reactive (donor) amino termini capable of forming isopeptide bonds with (acceptor) acidic residue side chains of up to four other proteins (i.e., BxpB, ExsY, CotY, or CotE) (Table 4). Second, like multiple BclA molecules capable of attaching to single BxpB molecule, multiple, and sometimes different, donor proteins (i.e., ExsY, CotY, or ExsB) were cross-linked to a single acceptor protein (i.e., BxpB, ExsY, CotY, or CotE) (FIGS. 8-10). Finally, the selection of acidic side chains of acceptor proteins is also promiscuous, but not random. Apparently, BxpB is divided into two domains that form isopeptide bonds with different donor proteins: the amino-terminal domain with ExsY/CotY and the rest domain of BxpB with BclA. Similarly, CotE also appears to be divided into two domains: a domain containing residues 1-154 available to form isopeptide bonds with multiple donor proteins and a smaller domain containing the last 26 residues of CotE (i.e., residues 155-180) that is shielded from isopeptide bond formation. As to the acceptor proteins ExsY and CotY, although there is no obvious division of domains like those of BxpB or CotE described above, only 8 of 15 acidic residues of ExsY, as well as 8 of 18 acidic residues of CotY, were observed to participate in isopeptide bond formation with a donor protein, suggesting a non-random selection of acidic side chains.

The present disclosure shows that CotE is directly cross-linked with multiple exosporium proteins (i.e., ExsY, CotY, or ExsB), indicating that at least some of CotE molecules are located in exosporium of B. anthracis. Given that CotE is essential for exosporium assembly and also plays a partial but significant role in coat protein assembly in B. anthracis (34), the present disclosure suggests that CotE is a morphogenetic protein located in the inner surface of basal layer, and perhaps also in other locations such as the coat or interspace. It is also noteworthy a proteolytic fragment containing only CotE sequence by the LC-MS/MS analysis was not identified, perhaps due to the huge amount of cross-links between CotE and other exosporium proteins.

Since BclA comprises the external hair-like nap, it is the outermost exosporium protein in the B. anthracis spore. As BclA is directly cross-linked to BxpB through the formation of isopeptide bonds, it is reasonable to infer that BxpB is located in the outer surface of basal layer. The results of this study also demonstrate that ExsY and CotY are required for the exosporium assembly of the >250-kDa complexes containing both BclA and BxpB, and that ExsY/CotY, as a donor or acceptor protein, is cross-linked with BxpB, ExsY, CotY, ExsB, or CotE via isopeotide bonds. This suggests that both ExsY and CotY are located throughout the entire basal layer and are interconnected with other exosporium proteins. In addition, ExsB is required for the stable exosporium attachment to the spore of B. anthracis and is cross-linked to ExsY, CotY, or CotE, but not BxpB, suggesting that ExsB is not near BxpB, perhaps located in the bottom half of the basal layer. Consistent with these suggestions for protein localization in the basal layer, BclA was not found to be cross-linked to ExsY, CotY, or CotE (data not shown).

The present disclosure suggests the following model for the exosporium protein network cross-linked by isopeptide bonds during exosporium assembly (FIG. 11). Following the synthesis, maturation (i.e., proteolytically processions for BclA, CotY, ExsY, and ExsB; phosphorylation for ExsB; glycosylation and trimerization for BclA), and proper incorporation of BclA, BxpB, ExsY, CotY, ExsB and CotE into the developing exosporium, isopeptide bonds are formed to cross-link a donor protein (i.e., BclA, ExsY, CotY, and ExsB) to an acceptor protein (i.e., BxpB, ExsY, CotY, and CotE). At the outer surface of basal layer, BclA trimers form isopeotide bonds with the entire region of BxpB except its amino-terminal domain, which is cross-linked by ExsY and/or CotY. ExsY/CotY, as either a donor or acceptor protein, also cross-links with other molecules of ExsY/CotY, ExsB, or CotE across the basal layer. Besides ExsY and CotY, ExsB also cross-links to CotE to stabilize the exosporium attachment. CotE, a morphogenetic protein located in the inner surface of basal layer (and perhaps also the coat or interspace), connects the exosporium to the coat of the spore directly or indirectly. It is also noteworthy that all of ExsY, CotY and ExsB are cysteine-rich proteins, which contain 12, 14, and 21 cysteines, respectively. Therefore, disulfide bonds might also be formed among these proteins during exosporium assembly.

Besides the six major structural proteins, other exosporium proteins might also be incorporated into this protein network through isopeptide bonds and/or disulfide bonds. One of them could be ExsK, which is also found to be tightly bound in the >250-kDa exosporium protein complexes (L. Tan and C. L. Turnbough, Jr., unpublished data). Furthermore, ExsK is another cysteine-rich exosporium protein with 12 cysteines in its 109 amino acids. Another candidate protein is ExsM, which appears to be proteolytically processed, although the manner of cleavage is unknown. B. anthracis strains lacking ExsM are encased in a double-layer exosporium, indicating that this protein plays a critical role in exosporium assembly. It is suggested that this complicated cross-linking protein network forms the framework for the entire exosporium assembly.

### Materials and Methods

### Bacterial Strains and Plasmids

The Sterne 34F2 avirulent veterinary vaccine strain of B. anthracis, obtained from the U.S. Army Medical Research Institute of Infectious Diseases, Fort Detrick, Md., was used as the wild-type strain and the parent in strain constructions. The Sterne stain is avirulent due to its inability to produce a capsule on vegetative cells; however, the exosporium of Sterne spores is essentially identical to the exosporium produced by virulent B. anthracis strains. Strain CLT304 (ΔrmlD) was a reconstruction of strain CLT274 (5). Strain CLT360 (ΔrmlD ΔbclA) was constructed by inserting the ΔbclA mutation from strain CLT292 (5) into the chromosome of strain CLT304 (ΔrmlD) by phage CP51-mediated generalized transduction (28). Construction of strain CLT307 (ΔbxpB) was previously described (10). Strain CLT325 (ΔexsY, Spec^{R}) was previously described (32). To construct the strain CLT298 (ΔcotY, Spec^{R}), codons 4 to 153 of 156 for the cotY gene in the WT strain was in-frame deleted, and a spectinomycin resistance cassette was inserted (using an engineered BamHI site) into an intergenic region 42 by upstream of the putative promoter of the cotY-bxpB operon, by allelic exchange essentially as previously described (30). To construct the double mutant strain CLT366 (ΔexsYΔcotY, Spec^{R} Kan^{R}), the same protocol except using a kanamycin resistant cassette was used to construct the cotY deletion in the genetic background of strain CLT325. All mutations were confirmed by PCR amplification of altered genetic loci and sequencing the DNA products.

Construction of the multi-copy plasmid pCLT1525, which encodes a BclA NTD-eGFP fusion protein expressed from the bclA promoter, was previously described (29). To construct plasmids expressing wild-type or mutant bxpB genes, the two-gene cotY-bxpB operon (i.e., promoter, genes, and transcription terminator) was inserted into the cloning site of multi-copy plasmid pCLT1474 (30). The DNA between the cotY-bxpB promoter region and the start codon of bxpB, including the entire cotY gene, was deleted by outward PCR (5). Up to 13 D/E to A point mutations were introduced into the wild-type bxpB gene of the recombinant plasmid by outward PCR. Each recombinant plasmid was introduced by electroporation into strain CLT307 (ΔbxpB). All mutations and constructions were confirmed by PCR amplification of altered genetic loci and sequencing the DNA products.

### Preparation of Spores and Exosporia

Spores were prepared by growing B. anthracis strains at 37° C. on LB agar plates until sporulation was complete, typically 3 to 4 days. Spores were washed from plates with cold (4° C.) sterile water (3 ml water per plate), collected by centrifugation. If needed, the obtained supernatant was saved and concentrated 10 times by speed vacuum. The spores in the pellet were further purified by sedimentation through a two-step gradient of 20% and 45% ISOVUE (Bracco Diagnostics), and washed extensively with cold sterile water. Spores were stored at 4° C. in sterile water and quantitated spectrophotometrically at 580 nm as previously described (31). Exosporia were purified from spores as previously described (9).

### Gel Electrophoresis and Immunoblotting

Spores (10⁸), exosporium samples, purified proteins, or the concentrated supernatants were boiled for 8 min in sample buffer containing 125 mM Tris-HCl (pH 6.8), 4% SDS, 100 mM dithiothreitol, 0.024% bromophenol blue, and 10% (v/v) glycerol. Solubilized proteins were separated by SDS-PAGE in a NuPAGE 4-12% Bis-Tris gel (Invitrogen). For immunoblotting, spore proteins were transferred from a polyacrylamide gel to a nitrocellulose membrane and detected by staining as previously described (9). Purified anti-BclA (EF-12), anti-BxpB (10-44-1) and anti-CotY/ExsY (G9-3) mouse MAbs were described previously (13), and the anti-GFP (GSN 149) mouse MAb was purchased from Sigma. Intensity of staining was measured by densitometry.

### Mass Spectrometry

For protein analysis by mass spectrometry, a Coomassie stained protein band was sliced from a polyacrylamide gel and digested with trypsin and chymotrypsin (15). Proteolytic fragments were analyzed by LC-MS/MS with electrospray ionization using a NanoLC Shimadzu pump linked to the Applied Biosystems 4000 Qtrap Mass Spectrometer. Interpretation of spectra was performed manually with the aid of the Analyst 1.4.2 software with BioAnalyst™ extensions.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein.

TABLE 1
BxpB fragments with attached AF peptides derived
from Bc1A

| BxpB Residues (SEQ ID | BxpB sequences with AF |
|---|---|
| NO: 2) | attachment site(s) in bold |
| 53-69 | ITVPVINDTVSVGDGIR |
| 60-69 | DTVSVGDGIR |
| 87-97 | DNSPVAPEAGR |
| 87-98 | DNSPVAPEAGRF |
| 92-97 | APEAGR |
| 118-134 | SNVIGTGEVDVSSGVIL |
| 118-134 | SNVIGTGEVDVSSGVIL |
| 145-157 | IVPVELIGTVDIR |

TABLE 2
BxpB fragments with attached AF peptides derived
from a Bc1A NTD-eGFP fusion protein

| BxpB Residues | BxpB sequence with AF | Band |
|---|---|---|
| (SEQ ID NO: 2) | attachment site(s) in bold | source |
| 1-10 | MFSSDCEFTK | 1 & 2 |
| 44-69 | LPSVSPNPNITVPVINDTVSV | 1 |
| | GDGIR | |
| 83-97 | TISLDNSPVAPEAGR | 2 |
| 83-98 | TISLDNSPVAPEAGRF | |
| 87-97 | DNSPVAPEAGR | 2 |
| 87-98 | DNSPVAPEAGRF | 2 |
| 87-98 | DNSPVAPEAGRF | 2 |
| 87-98 | DNSPVAPEAGRF | 2 |
| 118-134 | SNVIGTGEVDVSSGVIL | 2 |
| 118-137 | SNVIGTGEVDVSSGVILINL | 2 |
| 118-138 | SNVIGTGEVDVSSGVILINLN | 2 |
| 118-138 | SNVIGTGEVDVSSGVILINLN | 1 |
| 118-142 | SNVIGTGEVDVSSGVILINLN | 1 |
| | PGDL | |
| 120-137 | VIGTGEVDVSSGVILINL | 2 |
| 120-142 | VIGTGEVDVSSGVILINLNPG | 2 |
| | DL | |
| 138-144 | NPGDLIR | |
| 151-157 | IGTVDIR | 1 |

TABLE 3
rBxpB fragments with attached amino-terminal
peptides derived from rBc1A^{a}

| rBxpB Residues | rBxpB sequences with rBc1A |
|---|---|
| (SEQ ID NO: 2) | peptide attachment site(s) in bold |
| 1-10 | MFSSDCEFTK |
| 3-8 | SSDCEF |
| 3-8 | SSDCEF |
| 11-16 | IDCEAK |
| 11-24 | IDCEAKPASTLPAF |
| 11-26 | IDCEAKPASTLPAFGF |
| 45-69 | PSVSPNPNITVPVINDTVSVGD |
| | GIR |
| 60-69 | DTVSVGDGIR |
| 87-97 | DNSPVAPEAGR |
| 87-97 | DNSPVAPEAGR |
| 87-98 | DNSPVAPEAGRF |
| 92-97 | APEAGR |
| 118-134 | SNVIGTGEVDVSSGVIL |
| 118-138 | SNVIGTGEVDVSSGVILINLN |
| 138-144 | NPGDLIR |
| 145-157 | IVPVELIGTVDIR |
| 145-157 | IVPVELIGTVDIR |
| 151-157 | IGTVDIR |

^{a}Partial list showing 18 of 32 branched fragments.

TABLE 4
Isopeptide bonds formed in vivo between exosporium basal layer
proteins BxpB, CotY, ExsY, ExsB, and CotE.

| | | Reactive/ |
|---|---|---|
| | | total acidic |
| Donor protein | | residues |
| (amino-terminal | Acceptor protein (acceptor D/E | in acceptor |
| residue) | residue) | protein^{a} |
| CotY (S2) | BxpB (D5, 12/E7, 14) | 4/13 |
| CotY (S2) | CotY (D141/E71) | 2/18 |
| CotY (S2) | ExsY (D27, 89/E67, 86) | 4/15 |
| CotY (S2) | CotE (D61, 69, 85, 93, 99, 100/E3, | 17/38 |
| | 27, 46, 55, 57, 75, 79, 86, 115, 136, | |
| | 140) | |
| ExsY (S2) | BxpB (D5, 12/E7, 14) | 4/13 |
| ExsY (S2) | CotY (D141/E7, 71) | 3/18 |
| ExsY (S2) | ExsY (D17, 27, 89/E67, 86) | 5/15 |
| ExsY (S2) | CotE (D69, 99, 100/E6, 27, 31, 46, | 18/38 |
| | 55, 57, 75, 79, 86, 102, 115, 130, | |
| | 136, 140, 154) | |
| ExsB (E18) | CotY (D8, 13, 15, 95, 141/E7, 90) | 7/18 |
| ExsB (E18) | ExsY (D17, 27, 137/E24, 38) | 5/15 |
| ExsB (E18) | CotE (D93/E27, 46, 55, 57, 79, 115, | 8/38 |
| | 132) | |

^{a}Total numbers of D and E residues in acceptor proteins: BxpB (8D + 5E); ExsY (10D + 5E); CotY (12D + 6E); CotE (13D + 25E).

TABLE 5
BxpB fragments with attached amino-terminal peptides derived from ExsY/CotY

| BxpB residues | BxpB sequence with ExsY/CotY | Linking partner(s) |
|---|---|---|
| (SEQ ID NO: 2) | peptide attachment site(s) in bolds | of BxpB |
| 1-10 | MFSSDCE(CL1)FTK | ExsY/CotY |
| 2-8 | FSSD(CL1)CE(CL3)F | ExsY/CotY + CotY |
| 2-8 | FSSD(CL3)CEF | CotY |
| 2-8 | FSSDCE(CL4)F | ExsY |
| 3-8 | SSD(CL2)CEF | ExsY |
| 3-8 | SSD(CL2)CE(CL1)F | ExsY + ExsY/CotY |
| 3-10 | SSD(CL2)CE(CL4)FTK | 2 ExsY |
| 3-10 | SSD(CL4)CE(CL3)FTK | ExsY + CotY |
| 11-16 | ID(CL3)CEAK | CotY |
| 11-21 | ID(CL2)CE(CL3)AKPASTL | ExsY + CotY |
| 11-21 | IDCE(CL4)AKPASTL | ExsY |
| 11-28 | ID(CL4)CEAKPASTLPAFGFAF | ExsY |

a. CL, cross-linker, ExsY/CotY amino-terminal fragment cross-linked to a D/E residue of BxpB; CL1, SCN, ExsY/CotY common fragment; CL2, SCNEN, ExsY fragment; CL3, SCNCN, CotY fragment; CL4, SCNENK, ExsY fragment.

TABLE 6
ExsY/CotY fragments with attached amino-terminal peptides derived from another
ExsY/CotY

| ExsY (SEQ ID NO: 3)/ | | |
|---|---|---|
| CotY (SEQ ID NO: 4) | ExsY/CotY sequence with ExsY/CotY | Complex composition |
| residues^{a} | peptide attachment site(s) in bold^{b} | (donor + acceptor) |
| ExsY(57-75)/CotY(61-79) | ILYTKAGAPFE(CL1)AFAPSANL | ExsY/CotY + ExsY/CotY |
| ExsY(59-75)/CotY(63-79) | YTKAGAPFE(CL2)AFAPSANL | ExsY + ExsY/CotY |
| ExsY(67-79)/CotY(71-83) | E(CL1)AFAPSANLTSCR | ExsY/CotY + ExsY/CotY |
| ExsY(5-20) | ENKHHGSSHCVVD(CL2)VVK | ExsY dimer |
| ExsY(22-41) | INELQD(CL1)CSTTTCGSGCEIPF | ExsY/CotY + ExsY |
| ExsY(85-96) | VE(CL1)SVD(CL1)DDSCAVL | 2 × ExsY/CotY + ExsY |
| CotY(7-21) | E(CL2)DHHHHDCDFNCVSN | ExsY + CotY |
| CotY(131-145) | LISTNTCLTVD(CL3)LSCF | CotY dimer |
| CotY(132-145) | ISTNTCLTVD(CL4)LSCF | ExsY + CotY |

^{a}Numbers inside the bracket indicate the positions within ExsY/CotY of the amino acids included in the fragment.
^{b}CL, cross-linker, ExsY/CotY amino-terminal fragment cross-linked to a D/E residue of another ExsY/CotY; CL1, SCN, ExsY/CotY common fragment; CL2, SCNEN, ExsY fragment; CL3, SCNCN, CotY fragment; CL4, SCNENK, ExsY fragment.

TABLE 7
CotE fragments with attached amino-terminal peptides derived from ExsY/CotY

| CotE residues | CotE sequence with ExsY/CotY | Linking partner(s) |
|---|---|---|
| (SEQ ID NO: 6) | attachment site(s) in bold^{a} | of CotE |
| 1-5 | MSE(CL3)FR | CotY |
| 5-10 | RE(CL2)IITK | ExsY |
| 20-30 | TKSTHTCE(CL1)SNN | ExsY/CotY |
| 22-30 | STHTCE(CL2)SNN | ExsY |
| 31-39 | E(CL2)PTSILGCW | ExsY |
| 31-42 | E(CL2)PTSILGCWVIN | ExsY |
| 37-50 | GCWVINHSYE(CL3)ARKN | CotY |
| 40-52 | VINHSYE(CL1)ARKNGK | ExsY/CotY |
| 46-59 | EARKNGKEVE(CL1)IEGF | ExsY/CotY |
| 46-59 | E(CL1)ARKNGKHVEIE(CL1)GF | 2 × ExsY/CotY |
| 46-60 | E(CL2)ARKNGKHVEIEGFY | ExsY |
| 49-59 | KNGKHVE(CL1)IE(CL1)GF | 2 × ExsY/CotY |
| 50-59 | NGKHVE(CL3)IE(CL3)GF | 2 × CotY |
| 51-59 | GKHVE(CL2)IE(CL1)GF | ExsY + ExsY/CotY |
| 51-59 | GKHVE(CL3)IEGF | CotY |
| 51-60 | GKHVE(CL1)IEGFY | ExsY/CotY |
| 51-60 | GKHVEIE(CL2)GFY | ExsY |
| 53-59 | HVE(CL4)IE(CL2)GF | 2 × ExsY |
| 53-60 | HVE(CL3)IE(CL1)GFY | CotY + ExsY/CotY |
| 60-63 | YD(CL3)VN | CotY |
| 61-71 | DVNTWYSFD(CL4)GN | ExsY |
| 64-71 | TWYSFD(CL4)GN | ExsY |
| 69-83 | D(CL1)GNTKTEVVTE(CL1)RVNY | 2 × ExsY/CotY |
| 72-80 | TKTEVVTE(CL3)R | CotY |
| 72-83 | TKTE(CL1)VVTERVNY | ExsY/CotY |
| 74-83 | TE(CL1)VVTERVNY | ExsY/CotY |
| 83-91 | YTD(CL3)E(CL2)VSIGY | CotY + ExsY |
| 83-96 | YTDEVSIGYRD(CL3)KNF | CotY |
| 84-92 | TDE(CL1)VSIGYR | ExsY/CotY |
| 93-101 | D(CL3)KNFSGD(CL1)D(CL2) | LCotY + ExsY/CotY + |
| | | ExsY |
| 95-101 | NFSGD(CL1)D(CL3)L | ExsY/CotY + CotY |
| 96-101 | FSGD(CL2)D(CL3)L | ExsY + CotY |
| 96-101 | FSGDD(CL4)L | ExsY |
| 97-106 | SGDDLE(CL4)IIAR | ExsY |
| 115-121 | E(CL2)ALVSPN | ExsY |
| 115-123 | E(CL1)ALVSPNGN | ExsY/CotY |
| 115-123 | E(CL4)ALVSPNGN | ExsY |
| 115-124 | E(CL2)ALVSPNGNK | ExsY |
| 122-131 | GNKIVVTVE(CL2)R | ExsY |
| 132-142 | EFVTEVVGE(CL1)TK | ExsY/CotY |
| 134-142 | VTE(CL1)VVGE(CL1)TK | 2 × ExsY/CotY |
| 134-142 | VTEVVGE(CL2)TK | ExsY |
| 134-148 | VTE(CL1)VVGETKICVSVN | ExsY/CotY |
| 134-148 | VTE(CL4)VVGETKICVSVN | ExsY |
| 149-159 | PEGCVE(CL4)SDEDF | ExsY |

^{a}CL, cross-linker, ExsY/CotY amino-terminal fragment cross-linked to a D/E residue of CotE; CL1, SCN, ExsY/CotY common fragment; CL2, SCNEN, ExsY fragment; CL3, SCNCN, CotY fragment; CL4, SCNENK, ExsY fragment.

TABLE 8
ExsY fragments with attached EDF peptides derived
from ExsB

| ExsY residues | ExsY sequence with EDF |
|---|---|
| (SEQ ID NO: 3) | attachment site(s) in bold |
| 8-23 | HHGSSHCVVDVVKFIN |
| 8-25 | HHGSSHCVVDVVKFINEL |
| 24-42 | ELQDCSTTTCGSGCEIPFL |
| 26-42 | QDCSTTTCGSGCEIPFL |
| 128-147 | VSTSTCITVDLSCFCAIQCL |

TABLE 9
CotY fragments with attached EDF peptides derived
from ExsB

| CotY residues | CotY sequence with EDF |
|---|---|
| (SEQ ID NO: 4) | attachment site(s) in bold |
| 7-16 | EDHHHHDCDF |
| 7-17 | EDHHHHDCDFN |
| 7-21 | EDHHHHDCDFNCVSN |
| 88-100 | RVESIDDDDCAVL |
| 129-145 | ARLISTNTCLTVDLSCF |

TABLE 10
CotE fragments with attached EDF peptides derived
from ExsB

| CotE residues | CotE sequence with EDF |
|---|---|
| (SEQ ID NO: 6) | attachment site(s) in bold |
| 19-30 | YTKSTHTCESNN |
| 20-30 | TKSTHTCESNN |
| 43-52 | HSYEARKNGK |
| 46-59 | EARKNGKHVEIEGF |
| 72-80 | TKTEVVTER |
| 81-94 | VNYTDEVSIGYRDK |
| 113-124 | CLEALVSPNGNK |
| 132-142 | EFVTEVVGETK |

### REFERENCES

1. Mock, M., and A. Fouet. 2001. Anthrax. Annu. Rev. Microbiol. 55:647-671.
2. Henriques, A. O., and C. P. Moran, Jr. 2007. Structure, assembly, and function of the spore surface layers. Annu. Rev. Microbiol. 61:555-588.
3. Ball, D. A., R. Taylor, S. J. Todd, C. Redmond, E. Couture-Tosi, P. Sylvestre, A. Moir, and P. A. Bullough. 2008. Structure of the exosporium and sublayers of spores of the Bacillus cereus family revealed by electron crystallography. Mol. Microbiol. 68:947-958.
4. Boydston, J. A., P. Chen, C. T. Steichen, and C. L. Turnbough, Jr. 2005. Orientation within the exosporium and structural stability of the collagen-like glycoprotein BclA of Bacillus anthracis. J. Bacteriol. 187:5310-5317.
5. Daubenspeck, J. M., H. Zeng, P. Chen, S. Dong, C. T. Steichen, N. R. Krishna, D. G. Pritchard, and C. L. Turnbough, Jr. 2004. Novel oligosaccharide side-chains of the collagen-like region of BclA, the major glycoprotein of the Bacillus anthracis exosporium. J. Biol. Chem. 279:30945-30953.
6. Sylvestre, P., E. Couture-Tosi, and M. Mock. 2002. A collagen-like surface glycoprotein is a structural component of the Bacillus anthracis exosporium. Mol. Microbiol. 45:169-178.
7. Oliva, C., C. L. Turnbough, Jr., and J. F. Kearney. 2009. CD14-Mac-1 interactions in Bacillus anthracis spore internalization by macrophages. Proc. Natl. Acad. Sci. USA 106:13957-13962.
8. Oliva, C. R., M. K. Swiecki, C. E. Griguer, M. W. Lisanby, D.C. Bullard, C. L. Turnbough, Jr., and J. F. Kearney. 2008. The integrin Mac-1 (CR3) mediates internalization and directs Bacillus anthracis spores into professional phagocytes. Proc. Natl. Acad. Sci. USA 105:1261-1266.
9. Steichen, C., P. Chen, J. F. Kearney, and C. L. Turnbough, Jr. 2003. Identification of the immunodominant and other proteins of the Bacillus anthracis exosporium. J. Bacteriol. 185:1903-1910.
10. Steichen, C. T., J. F. Kearney, and C. L. Turnbough, Jr. 2005. Characterization of the exosporium basal layer protein BxpB of Bacillus anthracis. J. Bacteriol. 187:5868-5876.
11. Sylvestre, P., E. Couture-Tosi, and M. Mock. 2005. Contribution of ExsFA and ExsFB proteins to the localization of BclA on the spore surface and to the stability of the Bacillus anthracis exosporium. J. Bacteriol. 187:5122-5128.
12. Thompson, B. M., and G. C. Stewart. 2008. Targeting of the BclA and Bc1B proteins to the Bacillus anthracis spore surface. Mol. Microbiol. 70:421-434.
13. Tan, L., and C. L. Turnbough, Jr. 2010. Sequence motifs and proteolytic cleavage of the collagen-like glycoprotein BclA required for its attachment to the exosporium of Bacillus anthracis. J. Bacteriol. 192:1259-1268.
14. Redmond, C., L. W. Baillie, S. Hibbs, A. J. Moir, and A. Moir. 2004. Identification of proteins in the exosporium of Bacillus anthracis. Microbiology 150:355-363.
15. Kinter, M., and N. E. Sherman. 2000. The in-gel digestion protocol, p. 153-160, Protein sequencing and identification using tandem mass spectrometry. Wiley-Interscience, Inc.
16. Kang, H. J., and E. N. Baker. 2009. Intramolecular isopeptide bonds give thermodynamic and proteolytic stability to the major pilin protein of Streptococcus pyogenes. J. Biol. Chem. 284:20729-20737.
17. Alegre-Cebollada, J., C. L. Badilla, and J. M. Fernández. 2010. Isopeptide bonds block the mechanical extension of pili in pathogenic Streptococcus pyogenes. J. Biol. Chem. 285:11235-11242.
18. Marraffini, L. A., A. C. Dedent, and O, Schneewind. 2006. Sortases and the art of anchoring proteins to the envelopes of gram-positive bacteria. Microbiol. Mol. Biol. Rev. 70:192-221.
19. Wikoff, W. R., L. Liljas, R. L. Duda, H. Tsuruta, R. W. Hendrix, and J. E. Johnson. 2000. Topologically linked protein rings in the bacteriophage HK97 capsid. Science 289:2129-2133.
20. Kang, H. J., F. Coulibaly, F. Clow, T. Proft, and E. N. Baker. 2007. Stabilizing isopeptide bonds revealed in Gram-positive bacterial pilus structure. Science 318:1625-1628.
21. Ariëns, R. A., T. S. Lai, J. W. Weisel, C. S. Greenberg, and P. J. Grant. 2002. Role of factor XIII in fibrin clot formation and effects of genetic polymorphisms. Blood 100:743-754.
22. Kudryashov, D. S., Z. A. Durer, A. J. Ytterberg, M. R. Sawaya, I. Pashkov, K. Prochazkova, T. O. Yeates, R. R. Loo, J. A. Loo, K. J. Satchell, and E. Reisler. 2008. Connecting actin monomers by iso-peptide bond is a toxicity mechanism of the Vibrio cholerae MARTX toxin. Proc. Natl. Acad. Sci. USA 105:18537-18542.
23. Pickart, C. M. 2001. Mechanisms underlying ubiquitination. Annu. Rev. Biochem. 70:503-533.
24. Dierkes, L. E., C. L. Peebles, B. A. Firek, R. W. Hendrix, and R. L. Duda. 2009. Mutational analysis of a conserved glutamic acid required for self-catalyzed cross-linking of bacteriophage HK97 capsids. Virol. 83:2088-2098.
25. Osi\check{c}ka, R., K. Procházková, M. \check{S}ulc, I. Linhartová, V. Havlí\check{c}ek, and P. \check{S}ebo. 2004. A novel “clip-and-link” activity of repeat in toxin (RTX) proteins from gram-negative pathogens. Covalent protein cross-linking by an Asp-Lys isopeptide bond upon calcium-dependent processing at an Asp-Pro bond. J. Biol. Chem. 279:24944-24956.
26. Striebel, F., F. Imkamp, M. Sutter, M. Steiner, A. Mamedov, and E. Weber-Ban. 2009. Bacterial ubiquitin-like modifier Pup is deamidated and conjugated to substrates by distinct but homologous enzymes. Nat. Struct. Mol. Biol. 16:647-651.
27. Thompson, B. M., H. Y. Hsieh, K. A. Spreng, and G. C. Stewart. 2011. The co-dependence of BxpB/ExsFA and BclA for proper incorporation into the exosporium of Bacillus anthracis. Mol. Microbiol. 79:799-813.
28. Green, B. D., L. Battisi, T. M. Koehler, C. B. Thorne, and B. E. Ivins. 1985. Demonstration of a capsule plasmid in Bacillus anthracis. Infect. Immun. 49:291-297.
29. McPherson, S. A., M. Li, J. F. Kearney, and C. L. Turnbough, Jr. 2010. ExsB, an unusually highly phosphorylated protein required for the stable attachment of the exosporium of Bacillus anthracis. Mol. Microbiol. 76:1527-1538.
30. Dong, S., S. A. McPherson, L. Tan, O. N. Chesnokova, C. L. Turnbough, Jr., and D. G. Pritchard. 2008. Anthrose biosynthetic operon of Bacillus anthracis. J. Bacteriol. 190:2350-2359.
31. Nicholson, W. L., and P. Setlow. 1990. Sporulation, germination and outgrowth, p. 391-450. In C. R. Harwood and S. M. Cutting (ed.), Molecular biological methods for Bacillus. John Wiley & Sons, Ltd., West Sussex.
32. Boydston, J. A., L. Yue, J. F. Kearney, and C. L. Turnbough, Jr. 2006. The ExsY protein is required for complete formation of the exosporium of Bacillus anthracis. J. Bacteriol. 188:7440-7448.
33. Johnson, M. J., S. J. Todd, D. A. Ball, A. M. Shepherd, P. Sylvestre, and A. Moir. 2006. ExsY and CotY are required for the correct assembly of the exosporium and spore coat of Bacillus cereus. J. Bacteriol. 188:7905-7913.
34. Giorno R., et al. 2007. Morphogenesis of the Bacillus anthracis spore. J. Bacteriol. 189:691-705,
35. Driks, A., S. Roels, B. Beall, C. P. J. Moran, and R. Losick. 1994. Subcellular localization of proteins involved in the assembly of the spore coat of Bacillus subtilis. Genes Dev. 8:234-244.
36. Kim H., et al. 2006. The Bacillus subtilis spore coat protein interaction network. Mol. Microbiol. 59:487-502.
37. Todd, S. J., A. J. Moir, M. J. Johnson, and A. Moir. 2003. Genes of Bacillus cereus and Bacillus anthracis encoding proteins of the exosporium. J. Bacteriol. 185:3373-3378.

What is claimed:

1. A fusion protein, said fusion protein containing at least one donor sequence derived from a Bacillus species and a second polypeptide.

2. The fusion protein of claim 1, wherein the donor sequence is polypeptide sequence from a BclA, CotY, ExsY or ExsB polypeptide.

3. The fusion protein of claim 1, wherein the donor sequence is polypeptide sequence from a BclA polypeptide.

4. The fusion protein of claim 1, wherein the donor sequence is a full length BclA, CotY, ExsY or ExsB polypeptide.

5. The fusion protein of claim 1, wherein the donor sequence is a fragment of a full length BclA, CotY, ExsY or ExsB polypeptide.

6. The fusion protein of claim 1, wherein the donor sequence is a fragment of a full length BclA, CotY, ExsY or ExsB polypeptide, the fragment selected from the group consisting of: the first 40 amino acid residues, the first 38 amino acid residues, the first 20 amino acid residues, the first 10 amino acid residues, amino acid residues 2-40, amino acid residues 2-38 and amino acid residues 20-38, of the foregoing polypeptides.

7. The fusion protein of claim 1, wherein the second polypeptide of the fusion protein is taken from a polypeptide that is different from the polypeptide from which the donor sequence is derived.

8. A fusion protein, said fusion protein containing at least one acceptor sequence derived from a Bacillus species and a second polypeptide.

9. The fusion protein of claim 8, wherein the acceptor sequence is polypeptide sequence from a BxpB, CotE, CotY or ExsY polypeptide.

10. The fusion protein of claim 8, wherein the acceptor sequence is polypeptide sequence from a BxpB polypeptide.

11. The fusion protein of claim 8, wherein the acceptor sequence is a full length BxpB, CotE, CotY or ExsY polypeptide.

12. The fusion protein of claim 8, wherein the acceptor sequence is a fragment of a full length BxpB, CotE, CotY or ExsY polypeptide.

13. The fusion protein of claim 8, wherein the acceptor sequence is a fragment of a full length BxpB, CotE, CotY or ExsY polypeptide, the fragment selected from the group consisting of: a fragment at least 25 amino acids in length containing one or more acidic residues, a fragment at least 50 amino acids in length containing one or more acidic residues, a fragment at least 75 amino acids in length containing one or more acidic residues, a fragment at least 100 amino acids in length containing one or more acidic residues, a fragment at least 125 amino acids in length containing one or more acidic residues or a fragment at least 150 amino acids in length containing one or more acidic residues.

14. The fusion protein of claim 1, wherein the second polypeptide of the fusion protein is taken from a polypeptide that is different from the polypeptide from which the acceptor sequence is derived.

15. A method linking one or more polypeptides through a covalent bond, the method comprising the steps of:
a. providing a first polypeptide containing an acceptor sequence derived from a Bacillus species in a buffer;
b. providing a second polypeptide containing a donor sequence derived from a Bacillus species;
c. contacting the first and second polypeptides in the buffer in order to form a covalent bond between the acceptor sequence and the donor sequence, wherein the covalent bond is not a disulfide bond and the first polypeptide may optionally contain a donor sequence and the second polypeptide may optionally contain an acceptor sequence.

16. The method of claim 15 further comprising providing one or more additional polypeptides containing an acceptor sequence, a donor sequence or an acceptor sequence and a donor sequence.

17. The method of claim 15, wherein the acceptor sequence is polypeptide sequence from a BxpB, CotE, CotY or ExsY polypeptide and the donor sequence is polypeptide sequence from a BclA, CotY, ExsY or ExsB polypeptide.

18. The method of claim 15, wherein the acceptor sequence is a full length BxpB, CotE, CotY or ExsY polypeptide and the donor sequence is a full length BclA, CotY, ExsY or ExsB polypeptide or a fragment of a full length BclA, CotY, ExsY or ExsB polypeptide.

19. The method of claim 15, wherein the donor sequence is a fragment of a full length BclA, CotY, ExsY or ExsB polypeptide, the fragment selected from the group consisting of: the first 40 amino acid residues, the first 38 amino acid residues, the first 20 amino acid residues, the first 10 amino acid residues, amino acid residues 2-40, amino acid residues 2-38 and amino acid residues 20-38, of the foregoing polypeptides.

20. The method of claim 15, wherein the acceptor sequence is a full length BxpB, CotE, CotY or ExsY polypeptide or a fragment of a full length BxpB, CotE, CotY or ExsY polypeptide and the donor sequence is a full length BclA, CotY, ExsY or ExsB polypeptide.

21. The method of claim 15, wherein the acceptor sequence is a fragment of a full length BxpB, CotE, CotY or ExsY polypeptide, the fragment selected from the group consisting of: a fragment at least 25 amino acids in length containing one or more acidic residues, a fragment at least 50 amino acids in length containing one or more acidic residues, a fragment at least 75 amino acids in length containing one or more acidic residues, a fragment at least 100 amino acids in length containing one or more acidic residues, a fragment at least 125 amino acids in length containing one or more acidic residues or a fragment at least 150 amino acids in length containing one or more acidic residues.

22. The method of claim 15, wherein the acceptor sequence is a full length BxpB polypeptide or a fragment of a full length BxpB polypeptide containing one or more acidic residues and the donor sequence is a full length polypeptide BclA, CotY or ExsY polypeptide or a fragment of a full length BclA, CotY or ExsY polypeptide.

23. The method of claim 15, wherein the acceptor sequence is a full length ExsY polypeptide or a fragment of a full length ExsY polypeptide containing one or more acidic residues and the donor sequence is a full length polypeptide ExsY, CotY or ExsB polypeptide or a fragment of a full length ExsY, CotY or ExsB polypeptide.

24. The method of claim 15, wherein the acceptor sequence is a full length CotY polypeptide or a fragment of a full length CotY polypeptide containing one or more acidic residues and the donor sequence is a full length polypeptide ExsY, CotY or ExsB polypeptide or a fragment of a full length ExsY, CotY or ExsB polypeptide.

25. The method of claim 15, wherein the acceptor sequence is a full length CotE polypeptide or a fragment of a full length CotE polypeptide containing one or more acidic residues and the donor sequence is a full length polypeptide ExsY, CotY or ExsB polypeptide or a fragment of a full length ExsY, CotY or ExsB polypeptide.


SEQUENCE LISTING (26 sequences)

SEQ ID NO: 1 - protein, 400 aa, Bacillus anthracis
MSNNNYSNGLNPDESLSASAFDPNLVGPTLPPIPPFTLPTGPTGPTGPTGPTGPTGPTGPTGPTGPTGPTGPTGPTGPTGDTGTTGPTGPTGPTGPTGPTGDTGTTGPTGPTGPTGPTGPTGPTGPTGPTGPTGPTGDTGTTGPTGPTGPTGPTGPTGDTGTTGPTGPTGPTGPTGPTGPTGPTGPTGPTGPTGPTGPTGPTGDTGTTGPTGPTGPTGPTGPTGDTGTTGPTGPTGPTGPTGPTGPTGPTGATGLTGPTGPTGPSGLGLPAGLYAFNSGGISLDLGINDPVPFNTVGSQFGTAISQLDADTFVISETGFYKITVIANTATASVLGGLTIQVNGVPVPGTGSSLISLGAPIVIQAITQITTTPSLVEVIVTGLGLSLALGTSASIIIEKVA

SEQ ID NO: 2 - protein, 167 aa, Bacillus anthracis
MFSSDCEFTKIDCEAKPASTLPAFGFAFNASAPQFASLFTPLLLPSVSPNPNITVPVINDTVSVGDGIRILRAGIYQISYTLTISLDNSPVAPEAGRFFLSLGTPANIIPGSGTAVRSNVIGTGEVDVSSGVILINLNPGDLIRIVPVELIGTVDIRAAALTVAQIS

SEQ ID NO: 3 - protein, 152 aa, Bacillus anthracis
MSCNENKHHGSSHCVVDVVKFINELQDCSTTTCGSGCEIPFLGAHNTASVANTRPFILYTKAGAPFEAFAPSANLTSCRSPIFRVESVDDDSCAVLRVLSVVLGDSSPVPPTDDPICTFLAVPNARLVSTSTCITVDLSCFCAIQCLRDVTI

SEQ ID NO: 4 - protein, 156 aa, Bacillus anthracis
MSCNCNEDHHHHDCDFNCVSNVVRFIHELQECATTTCGSGCEVPFLGAHNSASVANTRPFILYTKAGAPFEAFAPSANLTSCRSPIFRVESIDDDDCAVLRVLSVVLGDTSPVPPTDDPICTFLAVPNARLISTNTCLTVDLSCFCAIQCLRDVTI

SEQ ID NO: 5 - protein, 186 aa, Bacillus anthracis
MKRDIRKAVEEIKSAGMEDFLHQDPSTFDCDDDCTTKIECSDDCNCPRTRCTRVKHCTFVTKCTHVKKWTFVTKCTRVRVQKWTFVTKVTRRKECVLVTKRTRRKHCTFVTKCVRFEKKFYWTKRCYCKKCEFFPHGHGGSCDDSCDHGKDCHDDGHKWNDCKGGHKFPSCKNKKFDHFWYKKRNC

SEQ ID NO: 6 - protein, 180 aa, Bacillus anthracis
MSEFREIITKAVVGKGRKYTKSTHTCESNNEPTSILGCWVINHSYEARKNGKHVEIEGFYDVNTWYSFDGNTKTEVVTERVNYTDEVSIGYRDKNFSGDDLEIIARVIQPPNCLEALVSPNGNKIVVTVEREFVTEVVGETKICVSVNPEGCVESDEDFQIDDDEFEELDPNFIVDAEEE

SEQ ID NO: 7 - protein, 19 aa, Bacillus anthracis
AFDPNLVGPTLPPIPPFTL

SEQ ID NO: 8 - protein, 14 aa, Bacillus anthracis
AFDPNLVGPTLPPI

SEQ ID NO: 9 - protein, 13 aa, Bacillus anthracis
FDPNLVGPTLPPI

SEQ ID NO: 10 - protein, 9 aa, Bacillus anthracis
AFDPNLPPI

SEQ ID NO: 11 - protein, 8 aa, Bacillus anthracis
FDPNLPPI

SEQ ID NO: 12 - protein, 9 aa, Bacillus anthracis
LVGPTLPPI

SEQ ID NO: 13 - protein, 8 aa, Bacillus anthracis
VGPTLPPI

SEQ ID NO: 14 - protein, 9 aa, Artificial Sequence
  artificial donor sequence
LVGPTLPPI

SEQ ID NO: 15 - protein, 8 aa, Artificial Sequence
  donor consensus sequence
VGPTLPPI

SEQ ID NO: 16 - protein, 29 aa, Bacillus anthracis
KRDIRKAVEEIKSAGMEDFLHQDPSTFDC

SEQ ID NO: 17 - protein, 20 aa, Bacillus anthracis
VEEIKSAGMEDFLHQDPSTF

SEQ ID NO: 18 - protein, 11 aa, Bacillus anthracis
KSAGMEDFLHQ

SEQ ID NO: 19 - protein, 19 aa, Bacillus anthracis
SCNENKHHGSSHCVVDVVK

SEQ ID NO: 20 - protein, 6 aa, Bacillus anthracis
SCNENK

SEQ ID NO: 21 - protein, 11 aa, Bacillus anthracis
SCNENKHHGSS

SEQ ID NO: 22 - protein, 15 aa, Bacillus anthracis
CNENKHHGSSHCVVD

SEQ ID NO: 23 - protein, 19 aa, Bacillus anthracis
SCNCNEDHHHHDCDFNCVS

SEQ ID NO: 24 - protein, 6 aa, Bacillus anthracis
SCNCNE

SEQ ID NO: 25 - protein, 11 aa, Bacillus anthracis
SCNCNEDHHHH

SEQ ID NO: 26 - protein, 16 aa, Bacillus anthracis
SCNCNEDHHHHDCDFN